(19) United States
(12) Patent Application Publication
(10) Pub. No.: US 2008/0313748 A1
(43) Pub. Date: Dec. 18, 2008

(54) Transgenic Non-Human Animals Comprising the Human Udp-Glucuronosyltransferase 1A (Ugt1a) Gene Locus and Methods of Using Them

(75) Inventor: Robert H. Tukey, San Diego, CA (US)

Correspondence Address: MORRISON & FOERSTER LLP, 12531 HIGH BLUFF DRIVE, SUITE 100, SAN DIEGO, CA, 92130-2040, US

(73) Assignee: Regents of the University of California, Oakland, CA (US)

(21) Appl. No.: 11/574,671

(22) PCT Filed: Sep. 2, 2005
(86) PCT No.: PCT/US05/31354

§ 371 Date: Nov. 16, 2007

Related U.S. Application Data
(60) Provisional application No. 60/607,239, filed on Sep. 3, 2004.

(51) Int. Cl. A01K 67/027; C12N 5/06; A01N 1/02; C12Q 1/02

(52) U.S. Cl. 800/3; 800/13; 800/14; 800/15; 800/16; 800/17; 800/18; 435/353; 435/354; 435/325; 435/1.1; 435/29

(57) ABSTRACT

The invention provides non-human transgenic animals, and cell lines, host cells, tissues and isolated organs, comprising the human UDP-glucuronosyltransferase IA (UGT1A) gene locus. In one aspect, the endogenous UGT1A gene locus of the non-human transgenic animal has been partially or completely “knocked out.” In another aspect, the invention is directed to drug screening, design and discovery. In another aspect, the invention is directed to determining the toxicity or metabolism of a compound, e.g., a toxin or drug, including environmental, dietary, cosmetic, biological warfare or other known or potentially toxic compounds. In another aspect, the invention is directed to deteuiining the toxicity or metabolism of a compound during a particular metabolic state of an animal, e.g., including pregnancy, stress, diet, age or a particular genotype.

### FEDERAL FUNDING

This invention was produced in part using funds from the Federal government under USPHS Grant Nos. ES10337 and GM49135. Accordingly, the Federal government has certain rights in this invention.

### TECHNICAL FIELD

This invention relates to molecular and cellular biology, biochemistry, molecular genetics, gene therapy, and drug design and discovery. In one aspect, the invention is directed to non-human transgenic animals and host cells comprising the human UDP-glucuronosyltransferase 1A (UGT1A) gene locus. In another aspect, the invention is directed to drug design or discovery.

### BACKGROUND

UDP-glucuronosyltransferases (UGTs) are a family of drug metabolizing enzymes contributing to hepatic drug metabolism and protection against environmental toxins. These enzymes function as the means to eliminate a variety of drug substances, environmental toxins, steroids and heme metabolites. Of significance is the fact that this particular locus is the most important in human drug metabolism. In rodents, while the locus is somewhat conserved, regulation of the locus is different. This means that when rodents are used by pharmaceutical or biotech firms for routine metabolism studies on potential new drug candidates, the results need to be extrapolated to the human. Most often, this can be done with relatively few surprises. Sometimes, however, because the UGT1A gene locus in the mouse is different from that in the human, there are unexpected results when moving drug development from rodent studies into human clinical trials.

The formation of β-glucopyranosiduronic acids by the multigene family of UDP-glucuronosyltransferases (UGTs) requires UDP-glucuronic acid to transform drugs and xenobiotics into hydrophilic glucuronides, converting the substrates into water soluble metabolites facilitating their excretion into the bile or urine. Located in the cellular endoplasmic reticulum, the UGTs play a vital role in the metabolism and detoxification of steroids, bile acids, hormones, environmental toxicants, carcinogens and a multitude of drugs.

In humans, the UGT1 and UGT2 gene families encode 19 RNA transcripts that have been identified from human tissues, and in vitro expression of these transcripts in tissue culture have aided in defining the substrate specificities of the UGTs. While UGT1 and UGT2 proteins are involved in drug metabolism, it is believed that the UGT1 proteins favor the metabolism of a greater proportion of xenobiotic substrates. Both UGT1 and UGT2 proteins participate actively in the glucuronidation of endobiotic substrates, with the UGT1 enzymes showing specificity for estrogens while the UGT2 proteins exhibit a preference for androgens as well as bile acids. Seven UGT2B genes and three UGT2A genes are encoded as individual structural genes on chromosome 4 and the UGT1 locus encodes 9 UGT1A proteins (UGT1A genes) on chromosome 2.

The UGT1A gene products are generated by a strategy of exon sharing, resulting in a family of microsomal proteins in which each contain a divergent amino terminal 280 amino acids and a commonly shared carboxy terminus that encodes 245 amino acids. The UGT1 locus spans more than 200 kb on chromosome 2 and is structured with a series of divergent exon 1 sequences that are organized consecutively over 150 kb with each exon 1 sequence encoding approximately 280 amino acids of the amino terminal portion. Located in the 3′ region of the locus are exons 2-5 which encode the conserved 245 amino acids of the carboxyl region. Flanking each of the exon 1 sequences are the necessary structural elements to assure appropriate transcriptional activation as monitored by expression in human tissue of UGT1A RNA gene transcripts. Reports regarding UGT1A RNA expression profiles indicate that each tissue contains a selective complement of UGT1A gene products with the gastrointestinal tract serving as a rich source for UGT1A expression. Adding to the uniqueness of these expression patterns, regulation of the UGT1 locus is also targeted by a number of xenobiotic and steroid receptors. The xenobiotic receptors pregnenolone X receptor (PXR) and the constitutive androstane receptor (CAR) as well as the Ah receptor have been shown in tissue culture to regulate UGT1A1 gene expression, promoting UGT1A1 protein induction. In addition, glucocorticoids work in a synergistic fashion to promote PXR and CAR induction of the UGT1A1 gene, providing support for the theory that circulating hormones may play a crucial role in maintaining appropriate levels of the UGTs in vivo. Exposure to selective environmental toxicants that activate the Ah receptor has been linked to transcriptional regulation of UGT1A6 and UGT1A9. Other Recent findings have also demonstrated that human variants of the PXR have been implicated in expression of UGT1A3 and UGT1A4, while the peroxisome proliferator-activated receptors (PPAR) α and β regulate UGT1A9. Thus, regulation of the UGT1 locus is believed to be controlled in a tissue specific manner by hormones, as well as by induction following exposure to xenobiotics.

Along with a uniquely divergent pattern of gene expression in human tissues, the UGT1A proteins comprise a compliment of proteins that are essential for the metabolism of most drugs. UGT1A dependent glucuronidation is an essential component of drug metabolism, and deficiencies in the ability to eliminate drugs through these processes can result in toxicities stemming from drug-drug interactions as well as pathological toxicities that are linked to heritable defects in the UGT1 locus. For example, there are more than 60 reported genetic lesions in the UGT1A1 gene that can lead to inheritable unconjugated hyperbilirubinemia. The most common in the human population is Gilbert's syndrome, which is associated with an altered promoter TATA sequence leading to reduced levels of UGT1A1. While Gilbert's syndrome is benign, adverse drug reactions have been linked to this reduction in UGT1A1 dependent glucuronidation. For example, the extreme toxicities associated with irinotecan therapy, a prodrug that is metabolized to SN-38 which then serves as potent topoisomerase inhibitor. Used conventionally in chemotherapy for solid tumors, SN-38 is metabolized by UGT1A1 and UGT1A7. Patients with Gilbert's syndrome are predisposed to hematological and gastrointestinal toxicities resulting from insufficient SN-38 glucuronidation. In addition, a TATA box polymorphism in the UGT1A7 promoter has been linked to reduced transcriptional activity, suggesting that reduced levels of UGT1A7 may be linked to adverse drug reactions associated with irinotecan therapy. A viable animal model to investigate the in vivo events associated with regulation of the UGT1A1 and UGT1A7 gene would be of considerable interest in furthering an understanding of the role of these proteins in adverse drug reactions.

One of the most important concepts in all of drug metabolism is an understanding of those events that control both infant and maternal drug metabolism during fetal and neonatal development. It is well known that levels of human glucuronidation gradually increase through development including the weeks and months following birth. Yet it might be anticipated that the dramatic changes in the levels of circulating hormones that occur during pregnancy and lactaction may alter the levels of hepatic enzymes in maternal liver. In rodents, several studies indicate that maternal liver glucuronidation activity is lower during pregnancy. However, in humans, selective glucuronidation activities during pregnancy are induced, as evident by increased oral clearance of paracetamol and lamotragine. Clearly, having available a “humanized” animal model to examine the impact of pregnancy on drug clearance would be a valuable tool in evaluating pharmacokinetic (PK) properties of therapeutic agents that are being developed for the use in humans.

### SUMMARY

The invention provides non-human transgenic animals and host cells, including tissues and organs, comprising the human UDP-glucuronosyltransferase 1A (UGT1A) gene locus and methods of using them. Thus, the invention provides animal models (and cells and tissues derived from them) and methods of using them for investigating and determining drug toxicity, drug detoxification, drug sensitivities (e.g., in different metabolic states, including any disease or condition, age, diet (including starvation or obesity), pregnancy or with various genotypes and phenotypes) and drug pharmacokinetics. The methods provided herein can be used to screen drugs in vivo and to design or discover drugs. In one aspect, the invention provides in vivo non-human animal, tissue, organ and cell models for assessing the toxicity, metabolism and/or pharmacokinetics of a composition or a compound, e.g., a drug, a small molecule, a polymer, a toxin, a steroid (e.g., a hormone), a heme metabolite, a cosmetic, a lotion, a food, a food or dietary supplement, an herbicide, a pesticide, a pollutant or a natural product. In one aspect, the composition or a compound tested (e.g., a toxin, drug) comprises an environmental toxin, a toxin derived from a natural product, a biological warfare agent or a toxin derived from a microorganism, or, a protein, a peptide, a nucleic acid, a carbohydrate, a polysaccharide, a fat, a steroid or a small molecule.

In one aspect, the animal models (and cells and tissues derived from them) of the invention are partially or completely “humanized” animal models, e.g., the corresponding endogenous UDP-glucuronosyltransferase 1A (UGT1A) gene locus has been partially or completely “knocked out”. Thus, the “humanized” animal models (and cells and tissues derived from them) of the invention can be used to examine the impact of pregnancy (or “pseudopregnancy) on the clearance of compounds, e.g., drug or toxin clearance. The “humanized” animal models of the invention can similarly be used to examine the impact of any particular genotype or phenotype, disease state, mental state (e.g., stress), environment (e.g., air or water pollution), diet (e.g., food or water contamination, high or low fat, starvation, obesity) and the like, on the clearance and/or metabolism of compounds. Thus, in one aspect the non-human animals, tissues, organs and cell models of the invention are used to evaluate pharmacokinetic (PK) properties of therapeutic agents that are being developed for the use in humans or other animals.

In one aspect, the endogenous UDP-glucuronosyltransferase 1A (UGT1A) gene locus of the non-human transgenic animal of the invention (comprising a functional human UGT1A gene locus) has been completely, or partially, disabled (“knocked out”). In one aspect, the invention provides a complete Ugt locus knock-out mouse comprising a functional human UGT1A gene locus. Thus, the invention also provides a non-human transgenic animal, e.g., a mouse, that is “humanized” with respect to the UDP-glucuronosyl-transferase 1A (UGT1A) gene locus. In this aspect, the invention provides an ins vivo animal model to evaluate the metabolism of a compound, e.g., a cosmetic, drug, lotion, food supplement, herbicide, pesticide, toxic pollutant, and the like. In one aspect, the compounds, e.g., drugs, toxins, etc, are glucuronidated, and these non-human transgenic animals (e.g., mice) are used to evaluate how drugs, toxins, etc. are cleared, and to relate this information to the behavior of drug metabolism in humans.

The invention is not limited to the “humanized” animal models; for example, an endogenous UDP-glucuronosyltransferase 1A (UGT1A) gene locus can be partially or completely “knocked out” in one non-human animal and replaced with an exogenous UGT1A gene locus from any other animal, including a human UGT1A gene locus.

By placing the UDP-glucuronosyltransferase 1A (UGT1A) gene locus into an in vivo environment that can now be targeted by tissue specific regulatory elements, the invention provides the compositions (cell and animal models, including a completely humanized UGT1A gene locus functions in a non-human animal model) and methods to examine the events involved in control of this locus. In one aspect, the invention provides compositions and methods to characterize the expression patterns of the human UGT1A locus genes and polypeptides in different tissues. Thus, the invention provides compositions and methods to analyze UGT1A locus gene and protein expression.

The invention provides non-human transgenic animals comprising a human UDP-glucuronosyltransferase 1A (UGT1A) gene locus. The non-human transgenic animal can be, e.g., a mouse. In one aspect, the endogenous UDP-glucuronosyltransferase 1A (UGT1A) gene locus of the non-human transgenic animal is completely or partially disabled (“knocked out”). The invention provides cells derived from the non-human transgenic animal of the invention. The invention provides cell lines derived from the non-human transgenic animal of the invention. The invention provides inbred mouse lines derived from the non-human transgenic animal of the invention. The invention provides inbred mouse lines comprising a human UDP-glucuronosyltransferase 1A (UGT1A) gene locus.

The invention provides methods of determining the pharmacokinetics or toxicity of a compound comprising: (a) providing a non-human transgenic animal of the invention; (b) providing a test compound; (c) administering the test compound to the animal; and (d) determining the pharmacokinetics or detoxification of a compound in the non-human transgenic animal. In one aspect, the test compound comprises a drug, an environmental toxin, a steroid, a heme metabolite, a cosmetic, a lotion, a food, a food or dietary supplement, an herbicide, a pesticide, a pollutant or a natural product.

Also provided herein are animal cells (e.g., human cells) comprising the human UDP-glucuronosyltransferase 1A (UGT1A) gene locus, e.g., as an episomal element, e.g., in an expression vector, or, as a heterologous insert stably inserted into the genome of the cell.

Also provided herein are kits including instructions for practicing the methods provided herein.

The details of one or more embodiments of the invention are set forth in the accompanying drawings and the description below. Other features, objects, and advantages of the invention will be apparent from the description and drawings, and from the claims.

All publications, patents, patent applications, GenBank sequences and ATCC deposits, cited herein are hereby expressly incorporated by reference for all purposes.

### DESCRIPTION OF DRAWINGS

FIG. 1 illustrates the identification of the UGT1 exons in mouse tail DNA by PCR, as described in detail in Example 1, below.

FIG. 2 is an illustration of a Western blot analysis of human UGT1A1, UGT1A4 and UGT1A6 identified in microsomes from liver, small intestine and large intestine from five Tg-UGT1 transgenic mouse founders, as described in detail in Example 1, below.

FIG. 3 illustrates data showing a differential regulation of the UGT1 gene locus in tissues from Tg-UGT^{1c} mice, as described in detail in Example 1, below.

FIGS. 4A and 4B illustrate an immunoblot analysis and resultant gene expression profiles of UGT1A1, UGT1A4 and UGT1A6 in Tg-UGT1^{1c} intestinal tissue following treatment with either pregenolone 16α-carbonitrile (PCN) or TCDD, as described in detail in Example 1, below.

FIG. 5 by illustration summarizes data showing induction of β-estradiol UGT activity in intestinal microsomes from PCN and TCDD treated Tg-UGT^{1c} mice, as described in detail in Example 1, below.

FIG. 6A is an illustration of an SDS-polyacrylamide gel electrophoresis separating samples of liver microsomal protein, and immunoblot analysis performed using UGT1A1-, UGT1A4 or UGT1A6-antibodies, as described in detail in Example 1, below. FIG. 6B is an illustration of electrophoresis in agarose gels showing total liver RNA which was used in reverse transcription reactions followed by PCR analysis, as described in detail in Example 1, below.

FIG. 7A top panel is an immunoblot of total cellular protein from primary hepatocytes from Tg-UGT^{1c} mice cultured in media that contained either 10 nM TCDD (T), 10 μM PCN (P) or 10 μM TCPOBOP (Tc) using the UGT1A1-antibody, followed by a Western blot of the same extracts using a CYP1A1-antibody, and in the bottom is an RT-PCR analysis of RNA extracted from these samples using specific oligonucleotide primers to detect the expression of mouse Cyp3a11, as described in detail in Example 1, below. FIG. 7B illustrates data summarizing the total RNA extracted from the different treatment groups using reverse transcription for Real Time PCR analysis of UGT1A1, as described in detail in Example 1, below.

FIG. 8 illustrates data from SDS-polyacrylamide gel electrophoresis and immunoblotting demonstrating maternal expression of UGT1A proteins during pregnancy and lactation, as described in detail in Example 1, below.

FIG. 9 illustrates the human UGT1A1 gene locus.

Like reference symbols in the various drawings indicate like elements.

### DETAILED DESCRIPTION

The invention provides non-human transgenic animals and host cells comprising a functional human UDP-glucuronosyltransferase 1A (UGT1A) gene locus and methods of using them. For example, the invention provides methods for determining the toxicity and pharmacokinetics of any compound, e.g., drugs, pesticides, herbicides, pollutants, and the like, using the cells and non-human animals (e.g., mice) of the invention.

The invention provides non-human transgenic animal models completely humanized for the UGT1A gene locus. In one aspect, the endogenous UDP-glucuronosyltransferase 1A (UGT1A) gene locus of the non-human transgenic animal of the invention (comprising a functional human UGT1A gene locus) has been completely, or partially, disabled (“knocked out”). In one aspect, the invention provides a complete Ugt locus knock-out mouse comprising a functional human UGT1A gene locus.

The invention provides non-human transgenic animal models, e.g., a transgenic mouse model, that carries the entire UGT1A locus, which is over 250 kb of DNA. The UGT1A locus regulation in the non-human transgenic animals and cells of the invention is similar to that seen in man. The transgenic mice of the invention are viable, and the expression patterns of the heterologous UGT1A gene locus has been characterized. For the first time, in non-human animal, e.g., rodents, one will be able to determine, and demonstrate, how compositions (e.g., drugs, pesticides, herbicides, pollutants, and the like) are cleared, imitating human drug metabolism.

Using the non-human transgenic animal and cell models, the invention provides methods to study those events that link homeostatic control of the UGT1 locus with various aspects of human glucuronidation in adult as well as during fetal development and lactation. For example, an exemplary mouse transgenic model that expresses a bacterial artificial chromosome encoding the entire UGT1 locus is described in detail in Example 1, below. Evidence is presented that each of the nine UGT1A genes is expressed in selective tissues. Thus, the non-human transgenic animals (e.g., in mice) and cell models of the invention can be used to study the expression of the UGT1 locus provides a unique opportunity to examine the regulatory properties that control not only the tissue specific and xenobiotic-receptor elicited expression patterns of the individual UGT1A genes, but enriches an understanding of how the UGT1 locus may be regulated at times where changes are apparent in the physiological levels of circulating hormones. The results described herein demonstrate that the non-human transgenic UGT1 animals (e.g., mice) and cell models of the invention can be effectively used for drug or toxicity screening and to investigate gene control of the UGT1 locus, and protein expression from the UGT1 locus, and to advance our understanding of how this locus is regulated in humans.

In one aspect, the UGT1 locus of the non-human transgenic and cell models of the invention encode 8 UGT proteins that are differentially expressed in an inducible and tissue specific fashion. Screening assays of the invention take into consideration the fact that individual tissues will display selective glucuronidation potential. Thus, cell lines of the invention (incorporating the human the UGT1 locus) can be derived from different tissues from non-human transgenic animals of the invention comprising the human the UGT1 locus, or alternatively from non-human transgenic animals and after isolation and culture have incorporated the human the UGT1 locus. Similarly, the endogenous UGT1 locus can be completely or partially disabled (“knocked out”) either before, during or after insertion of a human UGT1 locus. In one aspect, a stable inbred line of animals is generated and bred (e.g., a stable line of inbred mice having their endogenous UGT1 locus disabled, or “knocked out”) before the insertion of the human (or other animal's) UGT1 locus.

As discussed in detail in Example 1, below, examination of the factors that control UGT1 expression, BAC clones encoding the locus were identified and selective regulatory regions characterized. Through expression in tissue culture, the UGT1A1 gene was shown to bind functional AhR, PXR and CAR receptors in a region over −3500 bases from the promoter. A functional UGT1A1^{-3712/-7}-luciferase reporter construct was further analyzed for expression in transgenic mice. UGTLucR^{+/-} mice displayed little expression in liver and other extrahepatic tissues, with the exception of basal and AhR and PXR inducible expression in brain.

To examine if the lack of reporter activity resulted from the absence of important regulatory sequences needed for tissue specific expression, the exemplary transgenic mice of the invention expressing the entire UGT1 locus were used. Following characterization of several BAC clones encoding the locus, seven founder mouse lines expressing the UGT1 locus were generated. Mapping gene expression patterns by analysis of RNA encoding individual exon 1/exon 2 sequences, it was demonstrated that UGT1A1 was abundantly expressed throughout the gastrointestinal tract. Analysis of UGT1 gene expression patterns in UGT1^{+/-} mice confirmed that the locus is differentially regulated in a pattern concordant with previous observations made of UGT1 gene expression patterns in human tissues. These data demonstrate that the human UDP-glucuronosyltransferase 1A (UGT1A) gene locus in the non-human transgenic animals of the invention, particularly the exemplary transgenic mice, is regulated in a tissue and inducible specific fashion.

In one aspect, the invention provides a transgenic mouse model to study the expression patterns and inducibility of the human UGT1 locus. UGT1^{+/-} transgenic mice were developed following pro-nuclear injection of a human BAC clone encoding the locus. From forty-six initial founders, seven UGT1^{+/-} lines were characterized. Transmission of the UGT1 locus was followed through breeding experiments, and human specific primers for each gene were used to examine expression patterns in various tissues. Although multiple founders of the transgenic line transmit the entire locus to offspring, variations in patterns of basal expression among their offspring were observed in heart, lung, brain, and kidney. In the liver and other organs of the gastrointestinal tract, the transgenic expression was consistent among mice and mirrored the observed expression in humans. 1A7 is expressed in human stomach and 1A10 is expressed extrahepatically. This pattern of expression was also observed in the exemplary UGT1^{+/-} mice of the invention. Basal expression of 1A1, 1A3, 1A4, 1A6, and 1A9 was seen in liver, and 1A1, 1A3, 1A4, 1A6, and 1A10 in colon. Regulation of the human UGT1 locus is also maintained. When mice were treated with TCDD, elevated expression of 1A1 and 1A6 was observed in liver and small intestine, indicating that regulatory elements in the locus appear to be intact. Thus, the “humanized UGT1A gene locus” transgenic animal models (e.g., the mouse models) and cell lines of the invention are effective tools for studying the regulation and expression of human UGT1 genes (and the proteins they express) in a whole animal system. The “humanized UGT1A gene locus” transgenic animal models (e.g., the mouse models) and cell lines of the invention are effective tools and can be used to study and determine (and predict) the responsiveness of the human UGT1 locus (and thus the human) to agents such as drugs, cosmetics, dyes, cloth or fabric, chemicals, detergents, paints, toxins, poisons, biological warfare agents or any biological or synthetic chemical, e.g., industrial chemical, or natural product, and the like. Similarly, the transgenic animal models (e.g., the mouse models) and cell lines of the invention can be used to screen for agents capable of inducing activity of the human UGT1 locus—e.g., screening for agents that can be used to induce or boost an individual's ability to respond (e.g., detoxify by glucuronidation) to a drug, cosmetic, dye, fabric, chemical, detergent, paint, toxin, poison, biological warfare agent or any biological or synthetic chemical, e.g., an industrial chemical, or natural product, and the like.

Unless defined otherwise, all technical and scientific terms used herein have the same meaning as is commonly understood by one of skill in the art to which the invention(s) belong. All patents, patent applications, published applications and publications, Genbank sequences, websites and other published materials referred to throughout the entire disclosure herein are incorporated by reference in their entirety. In the event that there are a plurality of definitions for terms herein, those in this section prevail.

The term “gene” is used broadly to refer to any segment of nucleic acid associated with a biological function. Thus, genes include coding sequences and/or the regulatory sequences required for their expression. For example, “gene” refers to a nucleic acid fragment that expresses mRNA, functional RNA, or specific protein, including regulatory sequences. “Genes” also include non-expressed DNA segments that, for example, form recognition sequences for other proteins. “Genes” can be obtained from a variety of sources, including cloning from a source of interest or synthesizing from known or predicted sequence information, and may include sequences designed to have desired parameters. The term “gene” includes a nucleic acid sequence comprising a segment of DNA involved in producing a transcription product (e.g., a message), which in turn is translated to produce a polypeptide chain, or regulates gene transcription, reproduction or stability. Genes can include regions preceding and following the coding region, such as leader and trailer, promoters and enhancers, as well as, where applicable, intervening sequences (introns) between individual coding segments (exons). The term “genome” refers to the complete genetic material of an organism.

The term “transformation” refers to the transfer of a nucleic acid fragment into the genome of a host cell, resulting in genetically stable inheritance. A “host cell” is a cell that has been transformed, or is capable of transformation, by an exogenous nucleic acid molecule. Host cells containing the transformed nucleic acid fragments are referred to as “transgenic” cells, and organisms comprising transgenic cells are referred to as “transgenic organisms”. The terms “transformed”, “transduced”, “transgenic”, and “recombinant” refer to a host cell or organism into which a heterologous nucleic acid molecule has been introduced. The nucleic acid molecule can be stably integrated into the genome generally known in the art and are disclosed in Sambrook and Russell, infra. Known methods of PCR include, but are not limited to, methods using paired primers, nested primers, single specific primers, degenerate primers, gene-specific primers, vector-specific primers, partially mismatched primers, and the like. For example, “transformed,” “transformant,” and “transgenic” cells have been through the transformation process and contain a foreign gene integrated into their chromosome. The term “untransformed” refers to normal cells that have not been through the transformation process.

The terms “transfection of cells” refer to the acquisition by a cell of new nucleic acid material by incorporation of added DNA. Thus, transfection refers to the insertion of nucleic acid into a cell using physical or chemical methods. Several transfection techniques are known to those of ordinary skill in the art including: calcium phosphate DNA co-precipitation; DEAE-dextran; electroporation; cationic liposome-mediated transfection; and tungsten particle-facilitated microparticle bombardment (Johnston (1990). Strontium phosphate DNA co-precipitation is also a transfection method.

The terms “transduction of cells” refer to the process of transferring nucleic acid into a cell using a DNA or RNA virus. A RNA virus (i.e., a retrovirus) for transferring a nucleic acid into a cell is referred to herein as a transducing chimeric retrovirus. Exogenous nucleic acid material contained within the retrovirus is incorporated into the genome of the transduced cell. A cell that has been transduced with a chimeric DNA virus (e.g., an adenovirus carrying a cDNA encoding a therapeutic agent), will not have the exogenous nucleic acid material incorporated into its genome but will be capable of expressing the exogenous nucleic acid material that is retained extrachromosomally within the cell.

“Operably linked” as used herein refers to a functional relationship between two or more nucleic acid (e.g., DNA) segments. Typically, it refers to the functional relationship of transcriptional regulatory sequence to a transcribed sequence. For example, a promoter is operably linked to a coding sequence, such as a nucleic acid of the invention, if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

A “vector” comprises a nucleic acid which can infect, transfect, transiently or permanently transduce a cell. It will be recognized that a vector can be a naked nucleic acid, or a nucleic acid complexed with protein or lipid. The vector optionally comprises viral or bacterial nucleic acids and/or proteins, and/or membranes (e.g., a cell membrane, a viral lipid envelope, etc.). Vectors include, but are not limited to replicons (e.g., RNA replicons, bacteriophages) to which fragments of DNA may be attached and become replicated. Vectors thus include, but are not limited to RNA, autonomous self-replicating circular or linear DNA or RNA (e.g., plasmids, viruses, and the like, see, e.g., U.S. Pat. No. 5,217,879), and include both the expression and non-expression plasmids. Where a recombinant microorganism or cell culture is described as hosting an “expression vector” this includes both extra-chromosomal circular and linear DNA and DNA that has been incorporated into the host chromosome(s). Where a vector is being maintained by a host cell, the vector may either be stably replicated by the cells during mitosis as an autonomous structure, or is incorporated within the host's genome.

As used herein, the term “promoter” includes all sequences capable of driving transcription of a coding sequence in a cell, e.g., a plant cell or animal cell. Thus, promoters used in the constructs of the invention include cis-acting transcriptional control elements and regulatory sequences that are involved in regulating or modulating the timing and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) transcription. “Constitutive” promoters are those that drive expression continuously under most environmental conditions and states of development or cell differentiation. “Inducible” or “regulatable” promoters direct expression of the nucleic acid of the invention under the influence of environmental conditions or developmental conditions. Examples of environmental conditions that may affect transcription by inducible promoters include anaerobic conditions, elevated temperature, drought, or the presence of light.

“Tissue-specific” promoters are transcriptional control elements that are only active in particular cells or tissues or organs, e.g., in plants or animals. Tissue-specific regulation may be achieved by certain intrinsic factors which ensure that genes encoding proteins specific to a given tissue are expressed. Such factors are known to exist in mammals and plants so as to allow for specific tissues to develop.

The term “overexpression” refers to the level of expression in transgenic cells or organisms that exceeds levels of expression in normal or untransformed cells or organisms.

The term “plant” includes whole plants, plant parts (e.g., leaves, stems, flowers, roots, etc.), plant protoplasts, seeds and plant cells and progeny of same. The class of plants which can be used in the method of the invention is generally as broad as the class of higher plants amenable to transformation techniques, including angiosperms (monocotyledonous and dicotyledonous plants), as well as gymnosperms. It includes plants of a variety of ploidy levels, including polyploid, diploid, haploid and hemizygous states. As used herein, the term “transgenic plant” includes plants or plant cells into which a heterologous nucleic acid sequence has been inserted, e.g., the nucleic acids and various recombinant constructs (e.g., expression cassettes) of the invention.

“Plasmids” can be commercially available, publicly available on an unrestricted basis, or can be constructed from available plasmids in accord with published procedures. Equivalent plasmids to those described herein are known in the art and will be apparent to the ordinarily skilled artisan.

The phrases “nucleic acid” or “nucleic acid sequence” includes oligonucleotide, nucleotide, polynucleotide, or to a fragment of any of these, to DNA or RNA (e.g., mRNA, rRNA, tRNA) of genomic or synthetic origin which may be single-stranded or double-stranded and may represent a sense or antisense strand, to peptide nucleic acid (PNA), or to any DNA-like or RNA-like material, natural or synthetic in origin. The term encompasses nucleic acids, i.e., oligonucleotides, containing known analogues of natural nucleotides, naturally occurring nucleic acids, synthetic nucleic acids, and recombinant nucleic acids. The term also encompasses nucleic-acid-like structures with synthetic backbones, see e.g., Mata (1997) Toxicol. Appl Pharmacol. 144:189-197; Strauss-Soukup (1997) Biochemistry 36:8692-8698; Samstag (1996) Antisense Nucleic Acid Drug Dev 6:153-156.

The invention provides non-human transgenic animals comprising a complete UDP-glucuronosyltransferase 1A (UGT1A) gene locus. The UGT1A gene loci used to make or practice the invention can be operably linked to any heterologous sequences, e.g., cis-acting sequences, e.g., transcriptional regulators, such as promoters, intronic and exonic sequences, and the like. Promoters include, but are not limited to, any viral, bacterial or mammalian promoter, e.g., CMV immediate early, HSV thymidine kinase, early and late SV40, LTRs from retrovirus, and mouse metallothionein I, heat shock promoters, and LTRs from retroviruses. Other promoters known to control expression of genes in prokaryotic or eukaryotic cells or their viruses may also be used. The UGT1A gene loci used to make or practice the invention also can be operably linked to their endogenous transcriptional regulatory sequences, e.g., endogenous promoters, enhancers and the like. Endogenous transcriptional regulatory sequences can be modified by sequence variation, or their activity can be modified or manipulated by associate with other regulatory sequences.

In another aspect of the invention, a nucleic acid used to practice the invention, e.g., a UGT1A gene locus, an expression vector used to insert or express a UGT1A gene locus in a cell or a non-human transgenic animal, or any target sequence, can comprise a reporter or a marker gene (including nucleic acid sequences that encode proteins that can be used for reporting activity, e.g., enzymes or epitopes). In one aspect, the reporter or marker gene is used to monitor gene (e.g., UGT1A gene locus) expression, e.g., one, several or all coding sequence in the locus can be marked with the same or different markers. In one aspect, the reporter or marker gene is used to monitor gene suppression or silencing. In one aspect of the invention, the reporter gene comprises green fluorescent protein. Any compound, fluor, label, isotope, protein or gene that has a reporting or marking function can be used in the methods provided herein.

In another aspect of the invention, nucleic acids used to practice the invention, e.g., a UGT1A gene locus, an expression vector, or any target sequences are inserted into the genome of a host cell by e.g. a vector, a virus or any nucleic acid shuttling or insertional mechanism. For example, a nucleic acid sequence can be inserted into a genome or a vector by a variety of procedures. In one aspect, the sequence is ligated to the desired position in the vector following digestion of the insert and the vector with appropriate restriction endonucleases. Alternatively, blunt ends in both the insert and the vector may be ligated. In one aspect, viral long terminal repeats (LTRs) are inserted in a flanking pattern to effect insertion of a desired sequence (e.g., a UGT1A gene locus) into a genome. In one aspect, sequences homologous to a genome target sequence (targeting where in the genome it is desired to insert a desired nucleic acid, e.g., a UGT1A gene locus) are inserted in a flanking pattern to effect insertion of the desired sequence into a genome. A variety of cloning techniques are known in the art, e.g., as described in Ausubel and Sambrook. Such procedures and others are deemed to be within the scope of those skilled in the art.

The vector used to make or practice the invention can be chosen from any number of suitable vectors known to those skilled in the art, including cosmids, YACs (Yeast Artificial Chromosomes), megaYACS, BACs (Bacterial Artificial Chromosomes), PACs (P1 Artificial Chromosome), MACs (Mammalian Artificial Chromosomes), a whole chromosome, or a small whole genome. The vector also can be in the form of a plasmid, a viral particle, or a phage. Other vectors include chromosomal, non-chromosomal and synthetic DNA sequences, derivatives of SV40; bacterial plasmids, phage DNA, baculovirus, yeast plasmids, vectors derived from combinations of plasmids and phage DNA, viral DNA such as vaccinia, adenovirus, fowl pox virus, and pseudorabies. A variety of cloning and expression vectors for use with prokaryotic and eukaryotic hosts are described by, e.g., Sambrook. Particular bacterial vectors which can be used include the commercially available plasmids comprising genetic elements of the well known cloning vector pBR322 (ATCC 37017), pKK223-3 (Pharmacia Fine Chemicals, Uppsala, Sweden), GEM1 (Promega Biotec, Madison, Wis., USA) pQE70, pQE60, pQE-9 (Qiagen), pD10, psiX174 pBluescript II KS, pNH8A, pNH16a, pNH18A, pNH46A (Stratagene), ptrc99a, pKK223-3, pKK233-3, DR540, pRIT5 (Pharmacia), pKK232-8 and pCM7. Particular eukaryotic vectors include pSV2CAT, pOG44, pXT1, pSG (Stratagene) pSVK3, pBPV, pMSG, and pSVL (Pharmacia). However, any other vector may be used as long as it is replicable and viable in the host cell. In one aspect of the invention, target sequences are integrated into genomes using a lentiviral feline immunodeficiency (FIV) vector for the transduction process.

The invention provides non-human transgenic animals comprising a complete UDP-glucuronosyltransferase 1A (UGT1A) gene locus. In some aspects, the endogenous UGT1A gene locus has been completely, or partially, disabled (“knocked out”). Nucleic acids used to practice the invention, including the human UDP-glucuronosyltransferase 1A (UGT1A) gene locus, and vectors comprising this or other nucleic acids (e.g., including other UGT1A gene loci segments for making “knockout” animals) can be made, isolated and/or manipulated by, e.g., cloning and expression of cDNA libraries, amplification of message or genomic DNA by PCR, and the like. In practicing the methods of the invention, homologous genes (e.g., UGT1A loci genes) can be modified by manipulating a template nucleic acid, as described herein. The invention can be practiced in conjunction with any method or protocol or device known in the art, which are well described in the scientific and patent literature.

Non-human transgenic animals of the invention include both animals having stably inserted UGT1A sequences (e.g., a complete or partial human UDP-glucuronosyltransferase 1A (UGT1A) gene locus), unstable genomic inserts, mitochondrial inserts, or episomal inserts, e.g., as artificial chromosomes that are episomal to the endogenous chromosomes of the animal.

The nucleic acids used to practice this invention, whether RNA, iRNA, siRNA, antisense nucleic acid, cDNA, genomic DNA, vectors, viruses or hybrids thereof, may be isolated from a variety of sources, genetically engineered, amplified, and/or expressed/generated recombinantly. Recombinant polypeptides generated from these nucleic acids can be individually isolated or cloned and tested for a desired activity. Any recombinant expression system can be used, including bacterial, mammalian, yeast, insect or plant cell expression systems.

Alternatively, these nucleic acids can be synthesized in vitro by well-known chemical synthesis techniques, as described in, e.g., Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066. Alternatively, nucleic acids can be obtained from commercial sources.

Techniques for the manipulation of nucleic acids, such as, e.g., subcloning, labeling probes (e.g., random-primer labeling using Klenow polymerase, nick translation, amplification), sequencing, hybridization and the like are well described in the scientific and patent literature, see, e.g., Sambrook, ed., Molecular Cloning: A Laboratory Manual (2nd ed.), Vols. 1-3, Cold Spring Harbor Laboratory, (1989); Current Protocols in Molecular Biology, Ausubel, ed. John Wiley & Sons, Inc., New York (1997); Laboratory Techniques in Biochemistry and Molecular Biology: Hybridization with Nucleic Acid Probes, Part I. Theory and Nucleic Acid Preparation, Tijssen, ed. Elsevier, N.Y. (1993).

Another useful means of obtaining and manipulating nucleic acids used to practice the methods of the invention is to clone from genomic samples, and, if desired, screen and re-clone inserts isolated or amplified from, e.g., genomic clones or cDNA clones. Sources of nucleic acid used in the methods of the invention include genomic or cDNA libraries contained in, e.g., mammalian artificial chromosomes (MACs), see, e.g., U.S. Pat. Nos. 5,721,118; 6,025,155; human artificial chromosomes, see, e.g., Rosenfeld (1997) Nat. Genet. 15:333-335; yeast artificial chromosomes (YAC); bacterial artificial chromosomes (BAC); P1 artificial chromosomes, see, e.g., Woon (1998) Genomics 50:306-316; P1-derived vectors (PACs), see, e.g., Kern (1997) Biotechniques 23:120-124; cosmids, recombinant viruses, phages or plasmids.

In practicing the invention, nucleic acids of the invention or modified nucleic acids of the invention, can be reproduced by amplification. Amplification can also be used to clone or modify the nucleic acids of the invention. Thus, the invention provides amplification primer sequence pairs for amplifying nucleic acids of the invention. One of skill in the art can design amplification primer sequence pairs for any part of or the full length of these sequences.

Amplification reactions can also be used to quantify the amount of nucleic acid in a sample (such as the amount of message in a cell sample), label the nucleic acid (e.g., to apply it to an array or a blot), detect the nucleic acid, or quantify the amount of a specific nucleic acid in a sample. In one aspect of the invention, message isolated from a cell or a cDNA library are amplified.

The skilled artisan can select and design suitable oligonucleotide amplification primers. Amplification methods are also well known in the art, and include, e.g., polymerase chain reaction, PCR (see, e.g., PCR Protocols, A Guide to Methods and Applications, ed. Innis, Academic Press, N.Y. (1990) and PCR Strategies (1995), ed. Innis, Academic Press, Inc., N.Y., ligase chain reaction (LCR) (see, e.g., Wu (1989) Genomics 4:560; Landegren (1988) Science 241:1077; Barringer (1990) Gene 89:117); transcription amplification (see, e.g., Kwoh (1989) Proc. Natl. Acad. Sci. USA 86:1173); and, self-sustained sequence replication (see, e.g., Guatelli (1990) Proc. Natl. Acad. Sci. USA 87:1874); Q Beta replicase amplification (see, e.g., Smith (1997) J. Clin. Microbiol. 35:1477-1491), automated Q-beta replicase amplification assay (see, e.g., Burg (1996) Mol. Cell. Probes 10:257-271) and other RNA polymerase mediated techniques (e.g., NASBA, Cangene, Mississauga, Ontario); see also Berger (1987) Methods Enzyrnol. 152:307-316; Sambrook; Ausubel; U.S. Pat. Nos. 4,683,195 and 4,683,202; and Sooknanan (1995) Biotechnology 13:563-564.

### Cells and Tissues

The invention also provides cells and tissues (e.g., harvested from a non-human transgenic animal of the invention) comprising a complete or partial UGT1A gene loci, e.g., a human UGT1A gene loci. In one aspect of the invention, cells have gene expression that has been silences by mutation, sequence deletion, or by transcriptional silencing, e.g., where endogenous UGT1A loci genes are completely or partially silenced by mutation, sequence deletion and/or by transcriptional silencing. In one aspect, cells whose genes have been silenced, e.g., transcriptionally silenced, include plant and animal cells. In one aspect, animal cells include mammalian cells. In one aspect, the cell is a transgenic stem cell, e.g., a stem cell isolated from an animal of the invention, or, a transgenic stem cell made as described in U.S. Pat. No. 6,878,542.

Exemplary animal cells include CHO, COS or Bowes melanoma or any mouse or human cell line. The selection of an appropriate host is within the abilities of those skilled in the art.

Where appropriate, host cells can be cultured in conventional nutrient media modified as appropriate for activating promoters, selecting transformants or amplifying the genes of the invention. Following transformation of a suitable host strain and growth of the host strain to an appropriate cell density, the selected promoter may be induced by appropriate means (e.g., temperature shift or chemical induction).

Transgenic Non-Human Animals

The invention provides transgenic non-human animals comprising a complete or partial UGT1A gene loci, e.g., a human UGT1A gene loci, or subsequences thereof, including an expression cassette or vector or a transfected or transformed cell comprising a human UGT1A gene locus. The invention also provides methods of making and using these transgenic non-human animals.

The transgenic non-human animals can be any mammal, e.g., goats, rabbits, sheep, pigs, cows, cats, dogs, rats and mice, comprising a complete or partial UGT1A gene loci, e.g., a human UGT1A gene locus, or subsequences thereof. These animals can be used, e.g., as in vivo models to human UGT1A gene locus expression and activity, e.g., as models to screen for human UGT1A gene locus detoxifying activity in vivo, or to screen or compounds that can activate or depress UGT1A gene locus activity. The coding sequences for the polypeptides to be expressed in the transgenic non-human animals can be designed to be constitutive, or, under the control of tissue-specific, developmental-specific or inducible transcriptional regulatory factors. Transgenic non-human animals can be designed and generated using any method known in the art; see, e.g., U.S. Pat. Nos. 6,924,415; 6,825,395; 6,872,868; 6,211,428; 6,187,992; 6,156,952; 6,118,044; 6,111,166; 6,107,541; 5,959,171; 5,922,854; 5,892,070; 5,880,327; 5,891,698; 5,639,940; 5,573,933; 5,387,742; 5,087,571; 4,873,191; describing making and using transformed cells and eggs and transgenic mice, rats, rabbits, sheep, pigs and cattle (e.g., cows). For example, U.S. Pat. No. 6,872,868 describes genetic transformation of a zygote and the embryo and mature organism which result therefrom obtained by placing or inserting exogenous genetic material into the nucleus of the zygote or into any genetic material which ultimately forms at least a part of the nucleus of the zygote.

Transgenic non-human animals of the invention also can be designed and generated using methods as described, e.g., by Pollock (1999) J. Immunol. Methods 231:147-157, describing the production of recombinant proteins in the milk of transgenic dairy animals; Baguisi (1999) Nat. Biotechnol. 17:456-461, demonstrating the production of transgenic goats. U.S. Pat. No. 6,211,428, describes making and using transgenic non-human mammals which express in their brains a nucleic acid construct comprising a DNA sequence. U.S. Pat. No. 5,387,742, describes injecting cloned recombinant or synthetic DNA sequences into fertilized mouse eggs, implanting the injected eggs in pseudo-pregnant females, and growing to term transgenic mice whose cells express proteins related to the pathology of Alzheimer's disease. U.S. Pat. No. 6,187,992, describes making and using a transgenic mouse whose genome comprises a disruption of the gene encoding amyloid precursor protein (APP). U.S. Pat. No. 6,825,395, describes making transgenic pigs.

“Knockout animals” can also be used to practice the methods of the invention. For example, in one aspect, the transgenic or modified animals of the invention comprise a “knockout animal,” e.g., a “knockout mouse,” engineered not to express an endogenous gene, e.g., the endogenous UGT1A gene locus, or subsequences thereof. “Knockouts” can be prepared by deletion or disruption by homologous recombination of an endogenous promoter. “Knockout animals” or “Knockout cells” can be used to practice the methods of the invention. In one aspect, endogenous genes in stem cells are “knocked out” before insertion of a heterologous UGT1A gene locus. In alternative aspects, stem cells are myeloid, lymphoid, or neural progenitor or precursor cells. Stem cells may be derived from any vertebrate species, such as mouse, rat, dog, cat, pig, rabbit, human, non-human primates and the like. Homologous recombination and other means to alter (and “knockout”) expression of endogenous sequences is well known in the art and is described in, e.g., U.S. Pat. Nos. 5,464,764; 5,631,153; 5,487,992; 5,627,059; 5,272,071.

For example, in one exemplary method for making a transgenic non-human animal of the invention, an appropriate construct comprising all or part of a UGT1A gene locus is prepared. This construct is introduced into an appropriate host cell using any method known in the art, e.g., pronuclear microinjection; retrovirus mediated gene transfer into germ lines; gene targeting in embryonic stem cells; electroporation of embryos; sperm-mediated gene transfer; and calcium phosphate/DNA co-precipitates, microinjection of DNA into the nucleus, bacterial protoplast fusion with intact cells, transfection, polycations, e.g., polybrene, polyornithine, etc., or the like. In one aspect, the construct is introduced into an embryonic stem (ES) cells, which can be obtained from pre-implantation embryos cultured in vitro. These ES cells can be derived from an embryo or blastocyst of the same species as the developing embryo into which they are to be introduced. ES cells are typically selected for their ability to integrate into the inner cell mass and contribute to the germ line of an individual when introduced into the mammal in an embryo at the blastocyst stage of development See, e.g., any of the patents cited above.

If a regulated positive selection method is used in identifying homologous recombination events, the targeting construct is designed so that the expression of the selectable marker gene is regulated in a manner such that expression is inhibited following random integration but is permitted (de-repressed) following homologous recombination. In one aspect, transfected cells are screened for expression of a marker gene, e.g., the neo gene, which requires that (1) the cell was successfully electroporated, and (2) lac repressor inhibition of neo transcription was relieved by homologous recombination. This method allows for the identification of transfected cells and homologous recombinants to occur in one step with the addition of a single drug.

Alternatively, a positive-negative selection technique may be used to select homologous recombinants. This technique involves a process in which a first drug is added to the cell population, for example, a neomycin-like drug to select for growth of transfected cells, i.e. positive selection. A second drug, such as FIAU is subsequently added to kill cells that express the negative selection marker, i.e. negative selection. Cells that contain and express the negative selection marker are killed by a selecting agent, whereas cells that do not contain and express the negative selection marker survive. For example, cells with non-homologous insertion of the construct express HSV thymidine kinase and therefore are sensitive to the herpes drugs such as gancyclovir (GANC) or FIAU (1-(2-deoxy 2-fluoro-B-D-arabinofluranosyl)-5-iodouracil). See, e.g., Mansour (1988) Nature 336:348-352.

Selected cells can then injected into a blastocyst or other stage of development suitable for the purposes of creating a viable animal, e.g., a morula, of an animal (e.g., a mouse) to form chimeras (see e.g., Bradley, A. in Teratocarcinomas and Embryonic Stem Cells: A Practical Approach, E. J. Robertson, ed., IRL, Oxford, pp. 113-152 (1987)). Alternatively, selected ES cells can be allowed to aggregate with a dissociated animal embryo (e.g., mouse embryo) cells to form the aggregation chimera. A chimeric embryo can then be implanted into a suitable pseudopregnant female foster animal and the embryo brought to term. Chimeric progeny harboring the homologously recombined DNA in their germ cells can be used to breed animals in which all cells of the animal contain the homologously recombined DNA. In one aspect, chimeric progeny animals are used to generate an individual with a heterozygous disruption in a UGT1A gene locus. Heterozygous transgenic animals can then be mated. Typically ¼ of the offspring of such matings will have a homozygous disruption in the targeted gene. The heterozygous and homozygous transgenic animals can then be compared to normal, wild type individuals to determine whether disruption of the targeted gene causes phenotypic changes. For example, heterozygous and homozygous mice may be evaluated for phenotypic changes by physical examination, necropsy, histology, clinical chemistry, complete blood count, body weight, organ weights, and cytological evaluation of bone marrow.

The invention also provides conditional transgenic or knockout animals, e.g., animals produced using recombination methods. For example, an exemplary method comprises use of bacteriophage P1 Cre recombinase and flp recombinase from yeast plasmids. These are two non-limiting examples of site-specific DNA recombinase enzymes that cleave DNA at specific target sites (lox P sites for cre recombinase and frt sites for flp recombinase) and catalyze a ligation of this DNA to a second cleaved site.

### Drug Discovery

The methods and compositions of the invention can be used in drug discovery. The methods and compositions of the invention can be used for target validation; and, in some applications, can provide a physiologically accurate and less expensive approach to screen potential drugs. Expression arrays can be used to determine the expression of transgenic genes or genes other than a targeted gene or pathway.

The invention provides methods for determining the toxicity and pharmacokinetics of any compound, e.g., drugs, pesticides, herbicides, pollutants, and the like, using the cells and non-human transgenic animals of the invention.

### Kits and Libraries

The invention provides kits comprising compositions and methods of the invention, including cells, target sequences, transfecting agents, transducing agents, instructions (regarding the methods of the invention), or any combination thereof. As such, kits, cells, vectors and the like are provided herein.

The invention will be further described with reference to the following examples; however, it is to be understood that the invention is not limited to such examples.

### EXAMPLES

### Example 1

### Tissue Specific, Inducible, and Developmental Control of the Human UDP-Glucuronosyltransferase-1 (UGT1) Locus in Transgenic Mice

The following example describes making and using exemplary non-human transgenic mice of the invention.

Reagents: 2,3,7,8-Tetrachlorodibenzo-p-dioxin (TCDD) was obtained from Wellington Laboratories (Guelph, Ontario, Canada). Pregnenolone-16α-carbonitrile (PCN) and dexamethasone was obtained from Sigma, and 17-β-estradiol purchased from Calbiochem (San Diego, Calif.). 1,4-Bis-[2-(3,5-dichloropyridyloxy)]benzene (TCPOBOP) was from Sigma.

Generation of the UGT1 humanized mouse: A bacterial artificial chromosome encoding the entire human UGT1 locus described previously (e.g., in Yueh (2003) J. Biol. Chem. 278, 15001-15006) was purified by CsCl banding and dialyzed against microinjection buffer (10 mM Tris, pH 7.5, 0.1 mM EDTA, 30 μM spermine, 70 μM spermidine, and 100 mM NaCl). The purified DNA was microinjected into the pronucleus of CB6F₁ (an F1 hybrid between BALB/c and C57BL/6N mice) mouse eggs and transplanted into the oviduct of pseudopregnant C57BL/6N mice. All procedures for the generation of the transgenic mice were carried at the UCSD Superfund Transgenic Core Facility. For genotyping, DNA was isolated from tail clippings of 46-three week old mice and a 366-bp region in exon 5 of the common region of the human UGT1 locus was identified by PCR in 12 founders using sense (5′-cataaattaatcagccccag-3′, (SEQ ID NO:1) bases 187423-187443, AF297093) and antisense (5′-ccttctttaaacacacaagg-3′, (SEQ ID NO:2) bases 187789-187809) primers. Each founder was further profiled by PCR using specific primers that encoded a portion of each of the unique exon 1 sequences (Strassburg (1997) Mol. Pharmacol. 52, 212-220). Five founders containing the entire UGT1 locus were bred into C57B1/6N mice from Jackson Laboratory (Bar Harbor, Me.), and the F₁ offspring were used for further studies.

Preparation of antibodies to human UGT1A1, UGT1A4 and UGT1A6. The preparation of polyclonal antisera recognizing residues 29-159 of the human UGT1A1 protein has been described, e.g., by Ritter (1999) Hepatology 30, 476-484. Antisera recognizing human UGT1A4 and UGT1A6 were prepared using the same methodologies. Briefly, 6×-His-tagged fusion proteins were expressed in E. coli strain SG13009 (Qiagen) from pQE30 (Qiagen)-based plasmid constructs containing the coding sequence for residues 30-160 of UGT1A4 (construct pQE30-h1A4) or 12-131 of UGT1A6 (construct pQE30-h1A6). Expression of each fusion protein was induced in log phase cultures of transformed bacteria by addition of 1 mM isopropyl-B-D-thiogalactopyranoside (IPTG). After a 4 hour (h) induction, the cultures were harvested and fusion proteins were purified by affinity chromatography using Ni-NTA Sepharose affinity resin (Qiagen). Immunizations were performed using 10 female B6C3F1 mice for each individual form. One week after the final booster injection, animals were anesthetized and blood was collected by cardiac puncture. The protocol used for raising antisera followed NIH guidelines for the care and use of laboratory animals and received the approval of the Virginia Commonwealth University Institutional Animal Care and Use Committee. Serum samples for each antisera were pooled and aliquoted (50 μl/tube) prior to storage at −80° C.

Microsomal Protein Isolation from Transgenic Mouse Tissues. Using three animals per group, the liver, small and large intestinal tissues were collected from Tg-UGT1 and wild type mice. For the small and large intestine, the tissue was dissected open lengthwise and the luminal surface gently rinsed in 1.15% KCl before freezing on dry ice. Tissue samples from each treatment group were combined and frozen in liquid nitrogen in a porcelain mortar and pulverized under liquid nitrogen. A sample of the pulverized tissue was added to 5 volumes of 1.15% ice cold KCl and the tissue homogenized using a motorized glass-teflon homogenizer. The tissue homogenate was first centrifuged at 2,000×g for 10 min at 4° C. and the supernatant was collected. The supernatant was then centrifuged at 9,000×g for 10 min at 4° C. and this resulting supernatant centrifuged at 100,000×g for 60 min at 4° C. The pellet was resuspended in buffer (50 mM Tris-HCl pH 7.4, 10 mM MgCl₂, 1 mM PMSF) and the protein concentration determined by the Bradford method.

Immunoblot Analysis. All Western blots were performed using NuPAGE Bis-Tris polyacrylamide gels as outlined by the supplier (Invitrogen, Carlsbad, Calif.). Protein was heated at 70° C. for 10 min in loading buffer and resolved in 4-12% Bis-Tris gels under denaturing conditions (50 mM MOPS, 50 mM Tris-base, pH 7.7, 0.1% SDS, 1 mM EDTA) prior to transferring the proteins to polyvinylidene difluoride membrane using a semidry transfer system (Norvex, England). The membrane was blocked with 5% nonfat dry milk in 10 mM Tris-HCl, pH7.4 containing 0.15 M NaCl and 0.05% Tween 20 (Tris-buffered saline) for 1 h at room temperature, followed by incubation with primary antibodies (mouse anti-human UGT1A1, UGT1A4 or UGT1A6) in Tris-buffered saline overnight at 4° C. Membranes were washed and exposed to horseradish peroxidase-conjugated secondary antibodies for 1 h at room temperature. Each membrane was again washed and the conjugated horseradish peroxidase was detected using the ECL plus Western blotting detection system (Amersham) and the proteins detected following exposure to X-ray film.

Isolation and treatment of mouse transgenic primary hepatocytes. Primary hepatocytes were isolated from 8-12-week old mice. Mice were anesthetized by isoflurane inhalation. The portal vein was cannulated and perfused with Hanks' balanced salt solution (Ca²⁺ free and Mg²⁺ free) containing 0.1 mM EGTA and 10 mM Hepes at pH 7.4 for 5 min at the rate of 7 ml/min. As soon as perfusion is started, the anterior vena cava is cut to allow continuous flow to proceed out of the liver. At this time, the perfusate was changed to a solution containing 20 μg/ml Liberase Blendzymes (Roche) that was dissolved in Hanks' balanced salt solution (with Ca²⁺ and Mg²⁺), and the perfusion continued for another 5 min. The liver was removed and the hepatocytes were isolated by mechanical dissection followed by filtration through a sterile 70-μm filter. The cells were immediately collected by centrifugation at 50×g for 30 sec, and then the washing was repeated in DMEM tissue culture media. Cell viability was examined by Trypan-blue exclusion, and experiments conducted only if viability exceeded 90%. The hepatocytes were then cultured in 6-well collagen-treated plates (Discovery Labware, Bedford, Mass.) in 3 ml of DMEM medium containing penicillin/streptomycin and supplemented with 10% fetal bovine serum. Three hours after plating, the medium was replaced with fresh medium. The hepatocytes were treated with various chemicals 24 h after seeding for further studies. For analysis of proteins by Western blot, hepatocytes were collected and lysed in a buffer containing 0.05 M Tris-HCl, pH 7.4, 0.15 M NaCl, 0.25% deoxycholic acid and 1% NP-40 with a complement of protease and phosphatase cocktail inhibitors (Sigma). After incubation of this mixture for 30 min on ice, the solubilized lysate was centrifuged for 20 min in a refrigerated Eppendorf centrifuge at 16,000×g. The supernatant was collected and used directly for Western blot studies.

Determination of UGT Catalytic Activity. β-estradiol was prepared in ethanol. Catalytic activities of 100 μg of microsomal protein isolated from small and large intestinal tissues were assayed in duplicate in 50 mM Tris-HCl pH 7.6, 10 mM MgCl₂, 0.08 μCi [¹⁴C]UDPGA (PerkinElmer, 313 mCi/mmol), 0.5 mM unlabelled UDPGA, 0.1 mg/ml phosphatidylcholine, 8.5 mM saccharalactone, and 500 μM β-estradiol in a final volume of 100 μl for 60 minutes at 37° C. Reactions were terminated by the addition of 100 μl of methanol followed by centrifugation at top speed for 15 minutes. A 100 μl sample of the quenched reaction was spotted onto pre-adsorbent area of the TLC plate and develop in n-butanol/acetone/acetic acid/water (35:35:10:20) to achieve separation. ¹⁴C-Labeled glucuronides were visualized with a STORM 820™ PhosphorImager (Molecular Dynamics/Amersham Biosciences). Silica gel in regions corresponding to the glucuronide bands were then scraped from the TLC plates, radioactivity measured by liquid scintillation counting, and specific catalytic activities were calculated in picomoles of glucuronide formed/mg of protein/min.

Total RNA preparation and analysis of RNA by Real Time RT-PCR. Primary hepatocytes still attached to the collagen coated plates were washed in cold PBS once, followed by the addition of 1 ml acidic phenol/quanidinium isothiocyanate solution (TRIZOL™, Invitrogen). After 3 min, the TRIzol™ was removed and 200 μl chloroform was added and the solution was vortexed for 15 sec. The solution was centrifuged at 11,000 rpm in a refrigerated Eppendorf centrifuge for 15 min, and the water phase removed. The RNA was precipitated by the addition of 500 μl isoproponol and collected by centrifugation, followed by washing with 75% ethyl alcohol. Using OMNISCRIPT™ Reverse Transcriptase (Qiagen, Valencia, Calif.), approximately 2 μg of total RNA was used for the generation of complementary DNA (cDNA) as outlined by the manufacturer in a total volume of 20 μl. Following synthesis of cDNA, 2 μl was used in Real-Time PCR reactions conducted with a QUANTITECT™M SYBR® Green PCR Kit (Qiagen, Valencia, Calif.) according to the manufacturer's protocol. For detection of human UGT1A1 RNA, the forward primer was 5′-aacaaggagctcatggcctcc-3′ (SEQ ID NO:3) and the reverse primer was 5′-gttcgcaagattcgatggtcg-3′ (M57899) (SEQ ID NO:4). For analysis of the mouse β-actin RNA, the forward primer was 5′-atggccactgccgcatcctc-3′ (SEQ ID NO:5) and the reverse primer was 5′-gggtacatggtggtaccacc-3′ (SEQ ID NO:6). The polymerase was activated at 95° C. for 10 min followed by 40 cycles of amplification which consisted of the following: 95° C. for 30 sec, 63° C. for 1 min followed by 72° C. for 45 sec. Amplification was followed by DNA melt at 95° C. for 1 min and a 41-cycle dissociation curve starting at 55° C. and ramping 1° C. every 30 seconds (s). The MX4000 Multiplex QPCR™ (Stratagene, La Jolla, Calif.) was programmed to take three fluorescence data points at the end of each annealing plateau. All PCR reactions were performed in triplicate. Human UGT1A1 C(t) values were normalized to mouse β-actin C(t) values [ΔC(t)]. Human UGT1A1 RNA was expressed as induction fold over vehicle-treated cells using the equation ratio=2^{-(ΔCtSample-ΔCtVehicle)}.

Analysis of UGT1 gene expression patterns by reverse transcription-PCR: For RNA isolation from transgenic and wild type (WT) mouse tissues, the tissues from three animals were combined as described in the methods that outline the preparation of microsomal proteins. After pulverizing in liquid nitrogen, approximately 100 mg of tissue was homogenized in 1 ml of TRIzol™ solution, and the RNA extracted following the manufacturer's instructions. For each reverse transcription reaction, 2 μg of total RNA was denatured by heating and cDNA synthesized in 20 μl using the Omniscript RT™ kit (Qiagen) according to the manufacturer's instructions. From this reaction, 2 μl of the cDNA reaction was employed in each PCR reaction. Each PCR reaction contained 0.2 μM of mouse β-actin primers, 0.4 μM of each of the UGT1A specific oligonucleotide primer pair (11;15;50), and 15 μl HOTSTART MASTERMIX™ (Qiagen) in a 30 μl reaction. For UGT1A1, UGT1A3, UGT1A4, UGT1A5, UGT1A6, UGT1A7, UGT1A9 and UGT1A10 the polymerase was activated at 95° C. for 15 minutes followed by 30 cycles of 95° C. for 30 sec, 63° C. for 30 sec, and 72° C. for 45 sec, and a final extension at 72° C. for seven minutes. For UGT1A8 PCR amplification, the polymerase was activated at 95° C. for 15 min followed by 30 cycles of 95° C. for 30 sec, 58° C. for 30 sec, and 72° C. for 45 sec, and a final extension at 72° C. for seven minutes. Analysis of expressed RNA included an antisense oligonucleotide specific for the common region that encoded exon 2, while all of the sense primers encoded a highly specific segment of each exon 1 sequence that allowed for the unique identification of each UGT1A RNA. PCR reactions were carried out in a PerkinElmer Life Sciences GENEAMP™ DNA thermocycler PCR system. Ten microliters of each PCR product was resolved on a 1.5% agarose gel containing 1 μg/ml ethidium bromide and photographed using Polaroid 665 positive/negative film (Polaroid, Cambridge, Mass.).

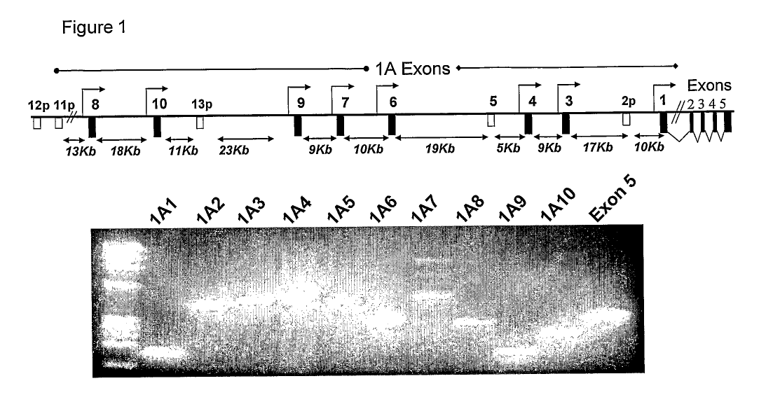

UGT1 locus expression in transgenic mice. The entire UGT1 locus was isolated from a human BAC genomic library and characterized by restriction enzyme mapping and DNA sequence analysis of the open reading frames (18). The locus extends in the 5′-direction, encoding all of the functional exon 1 sequences (1A1 through 1A10) as well as the conserved exons 2 through 5, see, e.g., Yueh (2003) supra. The BAC clone was purified and microinjected into fertilized FVB/N mouse eggs and transgenic mice were produced. Genotype analysis from tail clippings identified founders carrying exon 1 sequences UGT1A1 through UGT1A10 in addition to the 3′ non-coding region of exon 5, as illustrated in FIG. 1.

FIG. 1 illustrates the identification of the UGT1 exons in mouse tail DNA by PCR. The top drawing is a representation of the UGT1 locus and the organization of the unique 5′-exon 1 sequences and the conserved 3′-exons. The black boxes represent the unique exon 1 sequences (A1 through A10) which are spliced to common exons 2-5 which reside at the 3′ region of the locus. UGT1A13, UGT1A12, UGT1A11 and UGT1A2 are pseudogenes, and they are represented as open bars. PCR analysis of the human UGT1A sequences using tail DNA from Tg-UGT^{1c} mice is shown in the ethidium bromide stained gel following amplification of the sequences using human specific oligonucleotides that identify each of the exon 1 sequences (A1 through A10), as well as exon 5.

In addition, Southern blot analysis of genomic DNA from each of the exemplary transgenic lines of the invention showed hybridization signals that were the same as human genomic DNA, indicating that the linear arrangement of the UGT1 locus was structurally intact. Each of the transgenic founders was fertile and upon gross pathological examination they were indistinguishable from wild-type litter mates.

We arbitrarily selected five founders identified as Tg-UGT^{1a}, Tg-UGT^{1b}, Tg-UGT^{1c}, Tg-UGT1^{1d}, and Tg-UGT^{1e} for breeding experiments and all transmitted the UGT1 locus into F₁ progeny. Examination of the constitutive expression patterns of UGT1A genes was characterized by Western blot analysis to access the expression of UGT1A1, UGT1A4 and UGT1A6 in microsomal preparations from liver, small and large intestine. These experiments were performed with antibodies prepared against human UGT1A1 (as described by Ritter (1999) Hepatology 30, 476-484), UGT1A4 and UGT1A6. The polyclonal antibody to UGT1A1 has been shown previously not to react with rat liver microsomes (Ritter (1999) supra), and it does not recognize mouse Ugt proteins from liver microsomes. The UGT1A1, UGT1A4 and UGT1A6 antibodies are specific for these human isozymes as determined by Western blot analysis against each of the expressed proteins previously prepared in our laboratory. In the Tg-UGT1 mice, limited endogenous expression of human UGT1A1 was observed in liver, while UGT1A4 was identified in three founder lines and UGT1A6 clearly seen in two founder lines.

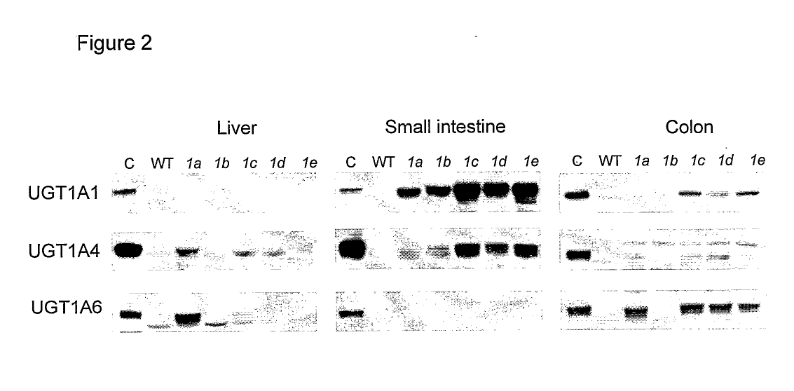

In preparing microsomes from the gastrointestinal tissue, small intestine preparations extended from the duodenum to the end of the ileum, and microsomes from the large intestine included the entire colon. In both small and large intestine, UGT1A1 was expressed, with the relative abundance being significantly higher in small intestine, as illustrated in FIG. 2, which is an illustration of a Western blot analysis of human UGT1A1, UGT1A4 and UGT1A6 identified in microsomes from liver, small intestine and large intestine from five Tg-UGT1 founders. Three mice representing each founder line along with wild type litters (WT) were used to prepare microsomes and samples (10 μg) of microsomal protein from liver, small intestine and large intestine were separated by SDS-polyacrylamide gel electrophoresis and transferred to nitrocellulose membranes. Specific UGT1A1-, UGT1A4- and UGT1A6-antibodies were used to identify expressed protein in these tissues. Included as an internal control for each blot are total cell extracts of expressed UGT1A1, UGT1A4 and UGT1A6 prepared from cDNAs that are stably expressed in HEK293 cells. The transgenic UGT1 founders are identified in the figure as Tg-UGT^{1a} (1a), Tg-UGT^{1b} (1b), Tg-UGT^{1c} (1c), Tg-UGT^{1d} (1d) and Tg-UGT^{1e} (1e).

As shown by the data illustrated in FIG. 2, the anti-human UGT1A4 antibody resolved a clear expression pattern in the small intestine from the five founders, with minimal but detectable expression in colon from four founder lines. Unlike UGT1A1 and UGT1A4, the expression of UGT1A6 was not observed in small intestinal microsomes. However, considerable expression of UGT1A6 was identified in colon microsomes from four Tg-UGT1 founders.

These results confirm that the UGT1 locus is functional in Tg-UGT1 mice with differences observed in tissue specific expression. Because consistent expression of UGT1A1, UGT1A4 and UGT1A6 was observed in founders Tg-UGT^{1a} and Tg-UGT^{1c}, we elected to proceed with a more thorough characterization of gene and protein expression in Tg-UGT^{1c}.

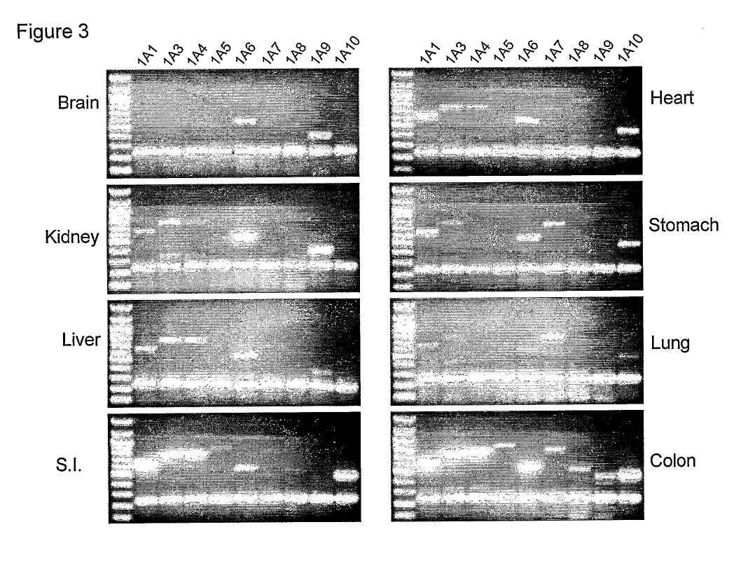

UGT1 expression patterns in tissues from Tg-UGT^{1c} mice. In experiments using human tissues, it has been demonstrated by RT-PCR that the UGT1 locus generates a pattern of gene expression that is unique for each tissue, see, e.g., Tukey (2000) Annu. Rev. Pharmacol. Toxicol. 40, 581-616; Tukey (2001) Molecular Pharmacology 59, 405-414. This type of analysis is possible by the use of highly specific oligonucleotides as primers to identify UGT1A gene expression patterns and is a useful tool in predicting tissue specific glucuronidation profiles, see, e.g., Strassburg (1997) supra; Strassburg (1999) Gastroenterology 116, 149-160. To illustrate the patterns of UGT1A expression in transgenic mouse tissues, a presentation of the RNA transcript patterns from Tg-UGT^{1c} are shown in FIG. 3, which illustrates data showing a differential regulation of the UGT1 gene locus in tissues from Tg-UGT^{1c} mice. UGT1 gene expression in different tissues was identified using isoform specific RT-PCR. RNA from each tissue was isolated from a pool of three tissues that were combined and pulverized in liquid nitrogen before RNA isolation in TRIzol (see Materials and Methods, above). The ethidium bromide stained gels show isoform-specific RT-PCR products co-amplified in the presence of β-actin primers as a control. Approximately 4 μg of RNA was used in each reverse transcription reaction before diluting the sample for each PCR reaction. In FIG. 3, PCR reactions were subjected to 30 extension cycles.

As illustrated in FIG. 3, low levels of UGT1A1, UGT1A3, UGT1A4 and UGT1A9 are observed in liver tissue with UGT1A6 being the most prominent. These five gene expression patterns have also been documented in human liver. UGT1A10, which was found expressed exclusively in extrahepatic tissues in human (see, e.g., Strassburg (1997) supra) is expressed in the gastrointestinal tract (small intestine, colon and stomach) of Tg-UGT^{1c} mice as well as in heart and lung tissue. UGT1A7, originally identified in human gastric epithelium (see, e.g., Strassburg (1997) supra), is found in transgenic stomach tissue, but is also predominantly expressed in lung. Expression in kidney from Tg-UGT1 mice is very selective with UGT1A6 and 1A9 RNA being the dominant forms identified, which also represent the expression patterns found in whole brain. In colon and small intestine, UGT1A1, UGT1A3 and UGT1A4 gene transcripts are abundant, while UGT1A6 is also abundant in colon.

The expression of UGT1A1, UGT1A4 and UGT1A6 as determined by immunoblot reflect RNA expression in these tissues, although a strict relationship between RNA abundance and protein accumulation is not necessarily maintained. For example, the relative levels of UGT1A1 RNA is comparable in small and large intestine, but the level of UGT1A1 as determined by Western blot analysis indicates a far greater accumulation of protein in the small intestine. Very little information is available that links UGT1A expression patterns to protein accumulation in human tissues, so the observed imbalance between RNA and protein abundance may indicate that UGT1A gene expression patterns may not be an accurate reflection of protein abundance in human tissues. It can also be noticed that UGT1A5 is found expressed in small and large intestine. This observation is of interest since the UGT1A5 gene product has not been cloned from human tissues. Like those results found in human colon (see, e.g., Strassburg (1998) J. Biol. Chem. 273, 8719-8726), gene transcripts representing each of the UGT1A proteins are detected in transgenic large intestine, indicating that a resemblance of human regulatory control is maintained in the transgenic mice.

Induction of the UGT1 locus by Ah receptor and PXR activators in the gastrointestinal tract. Several human UGTs have been shown to be regulated by activators of the Ah receptor (see, e.g., Yueh (2003) supra; Bock (1998) Adv. Enzyme Regul. 38, 207-222) and the pregnenolone X receptor (PXR) (see, e.g., Gardner-Stephen (2004) Drug Metab Dispos. 32, 340-347; Xie (2003) Proc. Natl. Acad. Sci. USA 100, 4150-4155). To look selectively at the induction of the UGT1 locus in Tg-UGT1 mice, Tg-UGT^{1c} mice were bred and three mice per group were selected for treatment with the either TCDD (16 μg/kg) or PCN (100 mg/kg). For both TCDD and PCN, the administration was by the intraperitoneal route, and each mouse was treated every 24 hours over a three day period. Tissues from three mice were then pooled and pulverized under liquid nitrogen, and samples used for microsomal preparation as well as for the isolation of total RNA.

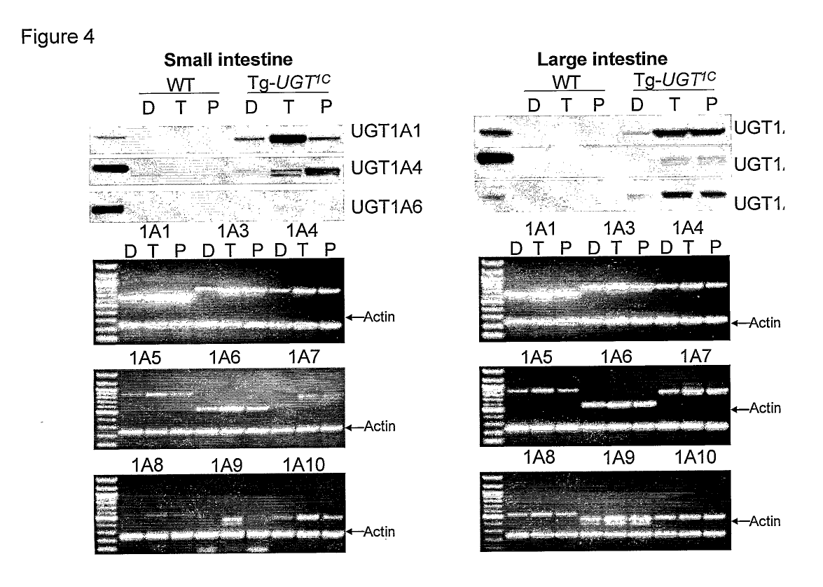

When we examined the levels of expressed UGT1A proteins in the gastrointestinal tract, defined induction patterns were observed. In small and large intestinal microsomal preparations, UGT1A1 was inducible by both TCDD and PCN, as illustrated in FIG. 4, which illustrates in immunoblot analysis and resultant gene expression profiles of UGT1A1, UGT1A4 and UGT1A6 in Tg-UGT1^{1c} intestinal tissue following treatment with either pregenolone 16α-carbonitrile (PCN) or TCDD. Three Tg-UGT^{1c} or wild type (WT) mice were treated by intraperitoneal injection every 24 hours with either DMSO, TCDD (16 μg/kg) or PCN (10 mg/kg) for 3 days. After 72 hours, the small intestines from each treatment group were combined and the large intestines from each treatment group were combined and the tissues pulverized under liquid nitrogen. A sample of each tissue was then used to prepare microsomes or to isolate total RNA. FIG. 4A: Western blot analysis of small and large intestinal microsomal protein using UGT1A1-, UGT1A4- or UGT1A6 specific antibodies. Included as control is a sample of each protein generated from the expression of cDNAs in stably transfected HEK293 cells. FIG. 4B: RNA prepared from the same samples of tissue were used in RT-PCR studies and the isoform specific products identified in ethidium bromide stained agarose gels.

The data illustrated in FIG. 4 demonstrate that the Ah receptor and PXR are functional in this tissue. This was consistent with previous findings demonstrating that UGT1A1 could be regulated by Ah receptor ligands (see, e.g., Yueh (2003) supra; Münzel (1998) Arch. Biochem. Biophys. 350, 72-78). Identification of Ah receptor enhancer sequences and evidence that the Ah receptor drives UGT1A1 transcription has been described in Yueh (2003) supra. Also identified in the enhancer region of the UGT1A1 gene were binding motifs that recognized PXR, which can be activated in rodents by PCN, see, e.g., Xie (2003) supra. In the gastrointestinal tract, UGT1A4 and UGT1A6 are differentially regulated, with UGT1A4 inducible in small and large intestine by both TCDD and PCN, while UGT1A6 appears to be predominantly regulated only in large intestine (see FIG. 4).

When we examined gene expression profiles, induction of all of the UGT1A gene transcripts was noted following treatment with either TCDD or PCN. Induction of UGT1A1 by TCDD and PCN in small and large intestine correlates with Western blot analysis of UGT1A1 in these tissues. Similar correlations can be made for both UGT1A4 and UGT1A6 in these tissues, although the abundance of UGT1A6 in small intestine as detected by immunoblot is not a good reflection of transcriptional activation. Interestingly, TCDD can be seen to induce all of the gene transcripts in either small or large intestine. Expression of UGT1A3 and UGT1A10 are particularly susceptible to induction in small intestine, with UGT1A5 and UGT1A7 being induced in large intestine.

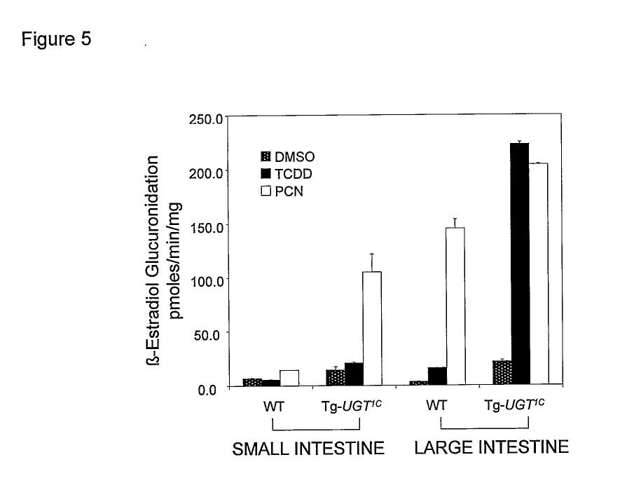

To determine if the expression of UGT1A gene products in Tg-UGT1^{1b} mice are active, gastrointestinal microsomes from the small and large intestine were prepared from untreated, PCN treated and TCDD treated WT and Tg-UGT1^{1c} mice and glucuronidation activity evaluated in microsomes using β-estradiol as substrate, as illustrated in FIG. 5, which summarizes data showing induction of β-estradiol UGT activity in intestinal microsomes from PCN and TCDD treated Tg-UGT^{1c} mice. The intestinal microsomal preparations generated in FIG. 4 were used to determine β-estradiol UGT activity, as outlined in Materials in Methods. Values are the mean±S.E.M from triplicate experiments.

Having demonstrated that UGT1A1 is induced by PCN and TCDD in the gastrointestinal tract, glucuronidation activity was evaluated with β-estradiol as a substrate, since this compound is an excellent substrate for analysis of expressed UGT1A1. In small and large intestinal microsomes isolated from WT and Tg-UGT^{1C} mice, β-estradiol glucuronidation activity was approximately 3 and 6 fold higher, respectively, in microsomes from untreated Tg-UGT^{1c} mice. Although PCN induced a minimal amount of UGT activity in small intestine from WT mice, β-estradiol glucuronidation activity was induced nearly 10 fold over those induced levels in WT mice. In large intestine, PCN induced β-estradiol glucuronidation significantly in both WT and TG-UGT^{1c} mice, yet the levels of activity were greater in the transgenics. The most significant induction of β-estradiol glucuronidation activity was observed in large intestinal microsomes from TCDD treated transgenic mice. Combined, these data indicate that elevated levels of UGT activity are the result of induction of the UGT1 locus by both TCDD and PCN in the gastrointestinal tract.

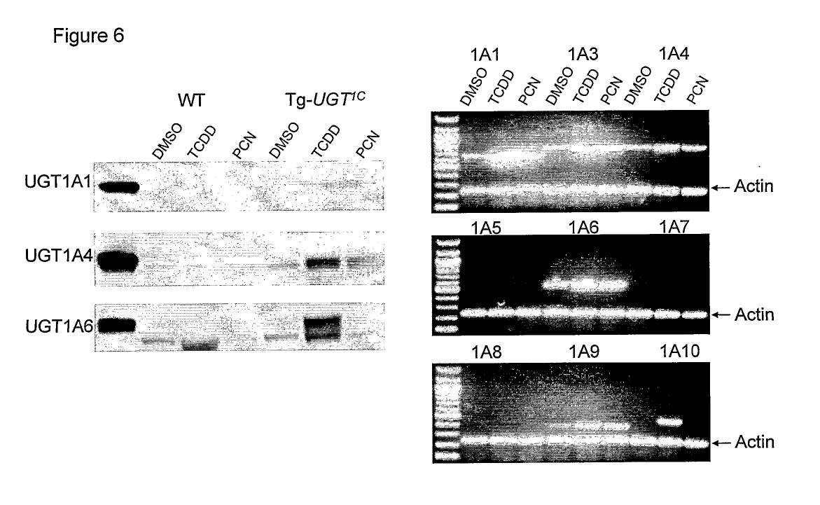

Induction of the UGT1 locus in liver by PCN and TCDD. When Tg-UGT^{1c} mice were treated with either TCDD or PCN, induction of microsomal UGT1A1, UGT1A4 and UGT1A6 was observed, as illustrated in FIG. 6, which illustrates data showing protein and gene expression patterns of UGT1A1, UGT1A4 and UGT1A6 in liver from Tg-UGT^{1c} mice treated with TCDD or PCN. Wild type and Tg-UGT^{1c} mice were treated every 24 hours with TCDD (16 μg/kg) or PCN (10 mg/kg) by intraperitoneal injection for 3 days, and the livers from three animals per group combined and used to prepare microsomes or to isolate total RNA. FIG. 6A: Samples of liver microsomal protein (10 μg) was separated by SDS-polyacrylamide gel electrophoresis, and immunoblot analysis performed using UGT1A1-, UGT1A4 or UGT1A6-antibodies. FIG. 6B: Total liver RNA (4 μg) was used in reverse transcription reactions followed by PCR analysis using isozyme-specific oligonucleotides. The transcripts were identified following electrophoresis in agarose gels stained with ethidium bromide.

When we examined gene expression profiles of the UGT1 locus by RT-PCR in Tg-UGT^{1c} liver, UGT1A1 RNA was present in untreated mice, but the antibody was unable to identify UGT1A1 protein in these mice. However, significant induction of UGT1A1 RNA was apparent following both TCDD and PCN treatment, a result that corresponded to induced UGT1A1 protein. The anti-UGT1A4 antibody recognized an endogenous protein in liver microsomes that migrates at approximately the same Rf value as human UGT1A4, but two bands can be seen in the sample isolated from PCN treated Tg-UGT^{1c} mice. It is apparent that the intensity of the antibody-recognized bands in Tg-UGT^{1c} untreated and TCDD treated mice is more intense than those in WT mice. An increase in UGT1A4 RNA is also visible in those samples taken from TCDD and PCN Tg-UGT^{1c} treated mice. The anti-UGT1A6 antibody recognizes a faster migrating protein in liver microsomes from WT mice that is clearly induced following TCDD treatment, and this protein may correspond to the mouse Ugt1a6. The induction pattern observed by RT-PCR confirms that UGT1A6 RNA is induced in TG-UGT^{1c} by PCN and TCDD, yet the levels of UGT1A6 protein are significantly greater in TCDD treated Tg-UGT^{1c} mice.

In human liver, a strict pattern of UGT1A1, UGT1A3, UGT1A4, UGT1A6 and UGT1A9 RNA expression has been observed (see, e.g., Strassburg (1997) supra; Strassburg (1999) supra). Analysis of UGT1A gene transcripts in Tg-UGT^{1c} liver demonstrates that both TCDD and PCN induce expression of each of these genes (see FIG. 6), indicating that they are targets for activated Ah receptor and PXR. This process is selective, since TCDD is shown to also induce UGT1A10 (see FIG. 6). The expression of UGT1A10 is not found constitutively in human liver, a finding which suggests that environmental exposure to Ah receptor ligands will lead to induction of this gene in liver. Since UGT1A10 is expressed in many extrahepatic tissues (see, e.g., Strassburg (1997) supra), its regulation is controlled by factors not present in liver. However, activation of the Ah receptor is sufficient to promote enhancer activity and transcriptional activation of the gene.

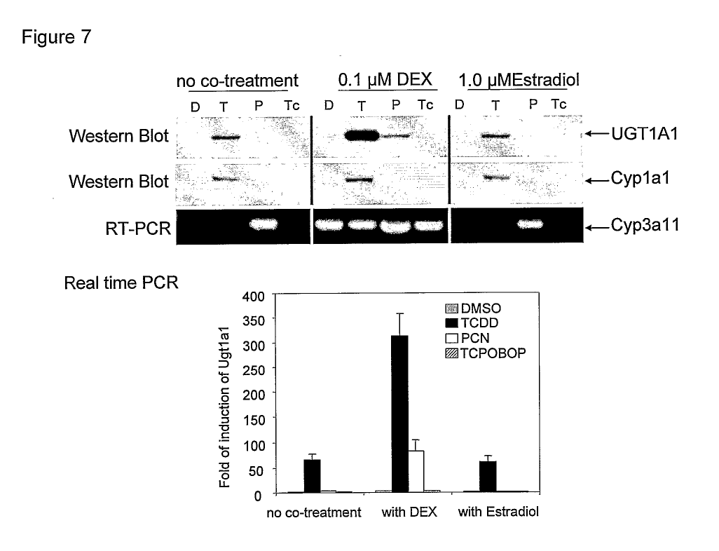

Reliance for glucocorticoids and the expression of UGT1A1 in primary hepatocytes. Expression of the UGT1 locus in liver led us to determine if induction patterns could also be observed in cultures of primary hepatocytes, as illustrated in FIG. 7, which illustrates the role of glucocorticoids in the expression of UGT1A1 in primary hepatocytes from Tg-UGT^{1c} mice. FIG. 7A: Primary hepatocytes from Tg-UGT^{1c} mice were cultured in media that contained either 10 nM TCDD (T), 10 μM PCN (P) or 10 μM TCPOBOP (Tc). Control hepatocyte cultures contained only DMSO (D). The same combination of treatments were conducted when hepatocytes also contained 0.1 μM dexamethasone or 1.0 μM β-estradiol. The top panel is an immunoblot of total cellular protein using the UGT1A1-antibody. This is followed by a Western blot of the same extracts using a CYP1A1-antibody. On the bottom is an RT-PCR analysis of RNA extracted from these samples using specific oligonucleotide primers to detect the expression of mouse Cyp3a11. FIG. 7B: total RNA extracted from the different treatment groups was used following reverse transcription for Real Time PCR analysis of UGT1A1. Tg-UGT^{1c} hepatocytes treated only with DMSO, TCDD, PCN or TCPOBOP are shown on the left, followed by analysis of hepatocytes co-treated with dexamethasone and either TCDD, PCN or TCPOBOP or hepatocytes co-treated with O-estradiol along with TCDD, PCN or TCPOBOP.

For these studies, hepatocytes isolated from Tg-UGT^{1c} mice were cultured on collagen coated petri dishes followed by analysis of expressed UGT1A1. The initial series of experiments demonstrated that hepatocytes treated with TCDD for 72 hours showed induction of UGT1A1 as well as mouse Cyp1a1, confirming that activation of the Ah receptor was sufficient to stimulate transcriptional activation of this gene. Interestingly, when hepatocytes were treated with PCN to activate PXR, limited induction of UGT1A1 was observed. In contrast, PXR activation by PCN was evident as shown by induction of Cyp3a11 mRNA.

It has been demonstrated that the glucocorticoid receptor (GR) and the glucocorticoid receptor-interacting protein (GRIP1) enhance PXR-mediated induction of UGT1A1 enhancer plasmid constructs, see, e.g., Sugatani (2005) Mol. Pharmacol. 67, 845-855. Dexamethasone has been shown to be a weak activator of PXR, but at a concentration of 0.1 μM dexamethasone, no induction of UGT1A1 in TG-UGT^{1c} isolated primary hepatocytes was observed. However, when hepatocytes were cultured in 0.1 μM dexamethasone and then treated with PCN, significant induction of UGT1A1 was observed. Interestingly, when primary hepatocytes from Tg-UGT^{1c} mice cultured in 0.1 μM dexamethasone were also treated with TCDD, UGT1A1 was induced 10 fold over the levels obtained only with TCDD treatment. The synergistic induction of UGT1A1 following treatment with TCDD or PCN may be a function of the glucocorticoid receptor, since these same increases do not occur when hepatoctyes are treated with β-estradiol, an activation of the estrogen receptor. Combined, these data indicate that induction of UGT1A1 requires the presence of circulating glucocorticoids or other humoral factors to elicit fall expression of the UGT1A1 gene.

Expression of the UGT1 locus during pregnancy. Considerable effort has been made to understand the role of glucucuronidation in neonatal development, see, e.g., Dutton, G. J. (1980) Glucuronidation of drugs and other compounds, CRC Press, Inc., Boca Raton, and it is well known in humans that bilirubin glucuronidation in newborns is induced immediately following birth. However, little information is known about the impact of fetal development or lactation on human glucuronidation. Since glucuronidation serves to detoxify and remove dietary and catabolic byproducts, it might be anticipated that dramatic changes in the levels of circulating hormones and other humoral factors resulting from fetal development and early neonatal life may impact the regulation and expression of maternal proteins that participate in xenobiotic metabolism. To examine this possibility, we undertook a series of experiments to quantitate the levels of hepatic UGT1A1, UGT1A4 and UGT1A6 in maternal Tg-UGT^{1c} mice at different stages during fetal development as well as during postnatal lactation and nursing.

Microsomes were prepared from pregnant Tg-UGT^{1c} mice every 7 days following mating and 7 and 14 days following birth. Immunoblot analysis of UGT1A1, UGT1A4 and UGT1A6 demonstrate that each of these proteins are induced in liver microsomes at 14 days postpartum. The expression of UGT1A1 returns to non-pregnant Tg-UGT^{1c} levels by birth, while the UGT1A4 and UGT1A6 levels remain slightly induced at 21 days. In maternal Tg-UGT^{1c} mice that are nursing, there is little change in the relative levels of hepatic UGT1A1 from those found in non-pregnant Tg-UGT^{1c} mice. However, tremendous induction of both UGT1A4 and UGT1A6 at 7 and 14 days following birth is demonstrated, indicating that hormonal balance during the period of lactation underlies this induction process. Combined, these data indicate that homeostatic control during fetal development and lactation play critical roles in the control and expression of the UGT1 locus.

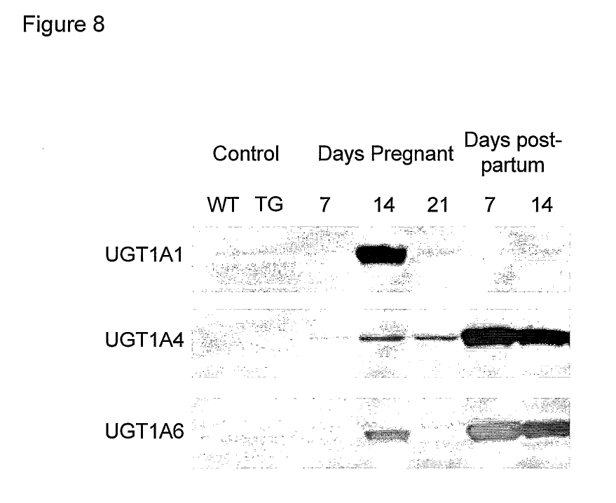

FIG. 8 illustrates data from SDS-polyacrylamide gel electrophoresis and immunoblotting demonstrating maternal expression of UGT1A proteins during pregnancy and lactation. Maternal microsomal proteins were prepared at 7, 14 and 21 days postpartum and 7, 14 and 21 days after birth. Neonates were present until microsomes were prepared from the nursing mothers. Aliquots of microsomes (15 μg) were subjected to SDS-polyacrylamide gel electrophoresis, and immunoblotting was performed with specific anti-UGT1A1, UGT1A4 and UGT1A6. Included in the Western blots were aliquots of liver microsomes from female wild type (WT) and non-pregnant Tg-UGT^{1c} mice.

### Discussion

These data demonstrate that the non-human transgenic animals and cells of the invention can express the UGT1 locus as in humans, including the nine proteins that actively participate in the metabolism of drugs as well as chemicals that come from environmental exposure. These data demonstrate that the non-human transgenic animals and cells of the invention can be used to gain an understanding of how the UGT1 locus is regulated in humans. This has was accomplished primarily from analysis of gene transcripts that can be identified in different tissues by identification of UGT1A RNA sequences. These studies using non-human transgenic animals and cells of the invention have been useful in categorizing the unique expression patterns in different tissues.

The identification of the sequences encoding each of the individual exon 1 regions and the flanking promoter regions has also been of value in attempting to determine in tissue culture those processes that might be important in controlling the tissue specific and potentially inducible patterns of expression of the human UGT1 proteins. It is known that UGT1A1, UGT1A6 and UGT1A9 can be regulated by chemicals that promote activation of the Ah receptor (see, e.g., Yueh (2003) supra; Bock (1998) supra), while UGT1A1, UGT1A3 and UGT1A4 are targets for xenobiotic receptors PXR or CAR (see, e.g., Sugatani (2001) Hepatology 33, 1232-1238; Gardner-Stephen (2004) supra; Xie (2003) supra). However, while these earlier studies were informative, an appropriate model to examine the tissue specific and inducible properties of the UGT1 locus and the functional outcome of these expression patterns has been lacking. The invention provides, and the data discussed herein demonstrates, that an exemplary transgenic animal model of the invention effectively expresses the UGT1 locus in a tissue specific and inducible pattern.

In the five founder strains that were examined, protein expression of UGT1A1, UGT1A4 and UGT1A6 were observed in liver and the gastrointestinal tract. Each of these proteins as well as their gene transcripts was found to be inducible by TCDD and PCN, demonstrating that glucuronidation in the liver and gastrointestinal tract can be subject to regulation by the Ah receptor and PXR. In liver and gastrointestinal tract, differences in the constitutive expression patterns of UGT1A4 and UGT1A6 was observed between the different founder lines. One possibility that may account for these differences in expression could be linked to the integration site of the BAC clone such that exposure of the chromatin to tissue-specific transcriptional factors is blocked. However, there was a consistent pattern of expression of UGT1A1 in both liver and the gastrointestinal tract in each of the founders. The inability to detect significant levels of UGT1A1 in liver microsomes may simply reflect minimal levels of protein expression, but detection of UGT1A1 RNA transcripts suggests that the UGT1A1 gene is regulated in liver. The importance of UGT1A1 in liver is crucial, since bilirubin is conjugated exclusively by UGT1A1 in humans, and is excreted into the bile through the basolateral surface of the hepatocytes to the biliary canniliculi. The lack of abundant liver UGT1A1 expression in rodents may be a reflection of diet, which in humans is felt to play an important role in the control and expression of UGT1A1 (see, e.g., Ishihara (2001) J. Gastroenterol. Hepatol. 16, 678-682; Tukey (2002) Mol. Pharmacol. 62, 446-450).

Alternatively, it is now speculated that species differences in the structure of the ligand-binding domain of the PXR provides selectivity in activation by endogenous activators such as species specific bile acids. It is conceivable that bile acid activation of rodent PXR is not sufficient to promote endogenous UGT1A1 transcriptional activation in Tg-UGT1 mice, but activation by other ligands may be sufficient to target gene induction of gene expression. There is support for this since activation of the rodent PXR can dominate transcriptional activation of UGT1A1 as demonstrated by PCN induction of UGT1A1 RNA in liver of Tg-UGT1 mice, see FIG. 6. Regardless, the data presented herein showing protein expression patterns in the liver and gastrointestinal tract demonstrate that the exemplary UGT1 transgenic mouse of the invention, and other non-human transgenic animals of the invention, are viable and accurate animal models to examine the expression patterns of the UGT1 locus in an intact animal model.

In liver, it was observed that UGT1A1, UGT1A3, UGT1A4, UGT1A6 and UGT1A9 were each subject to induction by both PCN and TCDD when gene transcript levels were examined by RT-PCR (FIG. 6). In the small and large intestine, PCN or TCDD treatment led to the induction of all nine of the UGT1A genes. The promotion of UGT1A gene transcription by TCDD in liver must require synergy with liver-specific transcriptional factors since UGT1A5, UGT1A7 and UGT1A8 are not regulated by TCDD in this tissue. This apparently is not the case in the induction of UGT1A10, where UGT1A10 is not expressed constitutively in Tg-UGT1 liver yet is significantly induced by TCDD. Although the expression of UGT1A10 has been considered to be exclusively an extrahepatic protein (see, e.g., Strassburg (1997) supra), this finding using an exemplary transgenic animal of the invention indicates that environmental exposure to Ah receptor ligands such as polycyclic aromatic hydrocarbons may promote the induction of UGT1A10 in human liver.

While a number of human tissues have been examined for the expression UGT1A5, this transcript has not been identified in humans (see, e.g., Tukey (2000) Annu. Rev. Pharmacol. Toxicol. 40, 581-616). In Tg-UGT^{1c} mice of the invention, UGT1A5 was found mildly expressed in small and large intestine and was also inducible following TCDD treatment. Induction of each of the UGT1A gene transcripts by TCDD links this process to activation of the Ah receptor, and must implicate binding of the Ah receptor/Arnt complex to enhancer xenobiotic receptor elements (XREs) (see, e.g., Gu (2000) Annu. Rev. Pharmacol. Toxicol. 40, 519-561). Ah receptor binding to XREs elements has been identified in the UGT1A1, UGT1A6 and UGT1A9 genes (see, e.g., Yueh (2003) supra; Munzel (1999) Drug Metab Dispos. 27, 569-573), and it might be anticipated that conserved XRE sequences are present on each of the UGT1A genes. However, it is certainly possible that a limited distribution of XRE sequences such as those located on the UGT1A1, UGT1A6 and UGT1A9 genes are sufficient as enhancer sequences to promote transcriptional activation of each of the UGT1A genes, since induction of UGT1A1, 1A3, 1A4, 1A5, 1A6, 1A7, 1A8, 1A9 and 1A10 RNA has been observed following TCDD treatment (see FIGS. 4 and 6).

In humans, the UGT1 locus is differentially regulated, with a unique complement of gene transcripts found in the different tissues (see, e.g., Tukey (2001) Molecular Pharmacology 59, 405-414). With the exception of liver and gastrointestinal tract, analysis of UGT1 expression patterns in other selective human tissues is lacking. When we examined expression patterns of the UGT1 locus in tissues from TG-UGT^{1c} mice of the invention, several of the expression patterns were similar to those found in human tissue. Tissue from human gastric epithelium highlighted the expression of UGT1A7 (11), a property of expression which is found in transgenic stomach. Interestingly, UGT1A7 is also found in abundance in transgenic lung tissue. This may be relevant since environmental toxicants such as polycyclic aromatic hydrocarbons present in tobacco smoke are substrates for UGT1A7 dependent glucuronidation (see, e.g., Strassburg (1998) supra; Zheng (2001) J. National Cancer Institute 93, 1411-1418), indicating that this protein may play an important first pass role in detoxifying these carcinogens in the lung. Although Zheng (2001)'s analysis of human lung did not identify UGT1A7, exposure to selective carcinogens such as polycyclic aromatic hydrocarbons and other Ah receptor activators may promote induction of the protein. All human liver samples that have been analyzed express UGT1A1, UGT1A3, UGT1A4, UGT1A6 and UGT1A9 (see, e.g., Strassburg (1997) supra; Strassburg (1999) supra), a pattern which is also maintained in transgenic liver. Human colon has been shown to express nearly all of the UGT1 gene transcripts (see, e.g., Strassburg (1999) supra), and this pattern is also maintained in transgenic large intestine. Certainly, the availability of a mouse model may be useful in predicting the expression patterns that may be found in human tissues. For example, TG-UGT^{1c} heart tissue expresses an abundance of the UGT1A transcripts implicating an important role for glucuronidation in this tissue. Using the transgenic animals and cells of the invention, we have identified UGT1A6 and UGT1A9 in whole brain, and this is relevant since it is known that selective neurotransmitters such as serotonin are subject to glucuronidation by UGT1A6 (see, e.g., King (1999) Arch. Biochem. Biophys. 365, 156-162). The tissue specific expression patterns found using transgenic animals and cells of the invention indicate regulation of the locus is under selective transcriptional control, a process that may be influenced by homeostatic control through circulating humoral factors.

In Tg-UGT^{1c} liver, UGT1A1 is induced following treatment with TCDD and PCN, indicating that cultured hepatocytes would be a viable tool to study the impact of UGT1A1 expression by xenobiotic receptor activation as well as the role of circulating hormones. When cultures of primary hepatocytes from Tg-UGT^{1c} mice were treated with TCDD, UGT1A1 was induced, a property that was reflected in activation of the Ah receptor and induction of mouse Cyp1a1. It is also apparent that PXR is activated following treatment of hepatocytes with PCN, since PXR targeted expression of Cyp3a11 RNA is observed. However, no induction of UGT1A1 is noted following Tg-UGT^{1c} hepatocyte treatment with PCN, indicating that additional regulatory factors are needed to support PCN elicited induction of this protein. Based upon the observation that glucocorticoids are weak activators of the PXR and may provide synergistic support for UGT1A1 expression, we noted that the addition of low concentrations (0.1 μM) of dexamethasone to the growth media facilitated PCN elicited induction of UGT1A1. Most notably, these low concentrations of dexamethasone supported over a 10-fold increase in TCDD induction of UGT1A1. The exaggerated induction of UGT1A1 by TCDD in the presence of glucocorticoids may be independent of Ah receptor function, since we did not observe a synergistic induction of Cyp1a1. This leads us to speculate that the synergistic induction of UGT1A1 by TCDD and PCN in the presence of dexamethasone may be occurring through a glucocorticoid receptor dependent mechanism that is working in concert with either the Ah receptor or the PXR. This result also suggests that circulating humoral factors may also participate in the regulation of the UGT1 locus.

Examining UGT1A expression profiles using the transgenic mice of the invention, we rationalized that the dramatic changes in steady-state levels of circulating hormones and steroid balance during pregnancy may provide an excellent opportunity to examine the impact of altered homeostatic control on maternal UGT1 expression. We observed that midway through gestation (day 14), expression of UGT1A1, UGT1A4 and UGT1A6 in liver was induced (FIG. 8), with the levels of expression returning to near normal levels just prior to birth. These results reflect findings that have been observed in clinical trials showing that drugs that are subject to glucuronidation by UGT1A4 and UGT1A6 are excreted at a greater rate during pregnancy (42) (59). Interestingly, these results are in contrast to findings in rats, where the levels of liver UGT1A1 were reduced in maternal liver during pregnancy (see, e.g., Luquita (2001) J. Pharmacol. Exp. Ther. 298, 49-56). We can interpret these results to suggest that the human UGT1A genes are controlled by activated regulatory factors resulting from hormonal changes and are linked to the early stages of fetal development, but rodent UGT1A genes lack this ability to be regulated during pregnancy. The contrasting results between human and rodent glucuronidation during pregnancy may be a reflection of differences in evolutionary conservation of selective cis-acting regulatory sequences on the human UGT1 and rodent UGT1 locus. The sharp increase in UGT1A glucuronidation capacity in maternal liver may also be a natural defense mechanism to facilitate detoxification or elimination of blood products resulting from catabolism during early embryogenesis.

The most dramatic UGT1A induction profile in maternal liver was observed with the induction of UGT1A4 and UGT1A6 following birth (FIG. 8). Interestingly, UGT1A1 was not induced relative to UGT1A4 and UGT1A6, indicating that selective humoral factors are modulating the regulation of UGT1A4 and UGT1A6 during lactation. Glucuronidation plays a critical role in the detoxification and removal of small lipophilic compounds and the dramatic induction of UGT1A4 and UGT1A6 may represent an example of the natural defense system that is activated during lactation assuring only the most essential nutrients be made available to the nursing neonates. There is support for this possibility since it has been demonstrated that lactating rats exhibit enhanced hepatic p-nitrophenol glucuronidation activity (see, e.g., Luquita (1994) Biochem. Pharmacol. 47, 1179-1185). We could also speculate that the induction of UGT1A4 and UGT1A6 during lactation is controlled through prolactin production, since it has been indicated that prolactin has been able to increase rat UGT1A6 but not rat UGT1A1 in ovariectomized rats (see, e.g., Luquita (2001) J Pharmacol. Exp. Ther. 298, 49-56). However, any one or a combination of the reproductive and metabolic hormones that are regulated during pregnancy and which impact on mammary gland development and lactation (e.g., as described in Neville (2002) J. Mammary. Gland. Biol. Neoplasia. 7, 49-66) may underlie the dramatic induction of UGT1A4 and UGT1A6.

Regardless, as demonstrated using the exemplary transgenic animals and cells of the invention as described herein, expression of the UGT1 locus in maternal tissue during pregnancy and lactation appears to undergo significant regulation, an observation which indicates that maternal glucuronidation plays a critical role in fetal and neonatal development. These findings suggest that one of the key actions of hormones or other humoral factors during pregnancy and neonatal development is to serve as a signal in the maternal circulation to provide a means for robust detoxification pathways. Along with other observations that the UGT1 locus is a target for regulation by xenobiotics in combination with tissue specific events, this exemplary transgenic mouse model of the invention, in addition to all transgenic animal models of the invention, are useful to study the impact of UGT1A metabolism on selective drugs as a function of induction and development.

A number of embodiments of the invention have been described. Nevertheless, it will be understood that various modifications may be made without departing from the spirit and scope of the invention. Accordingly, other embodiments are within the scope of the following claims.

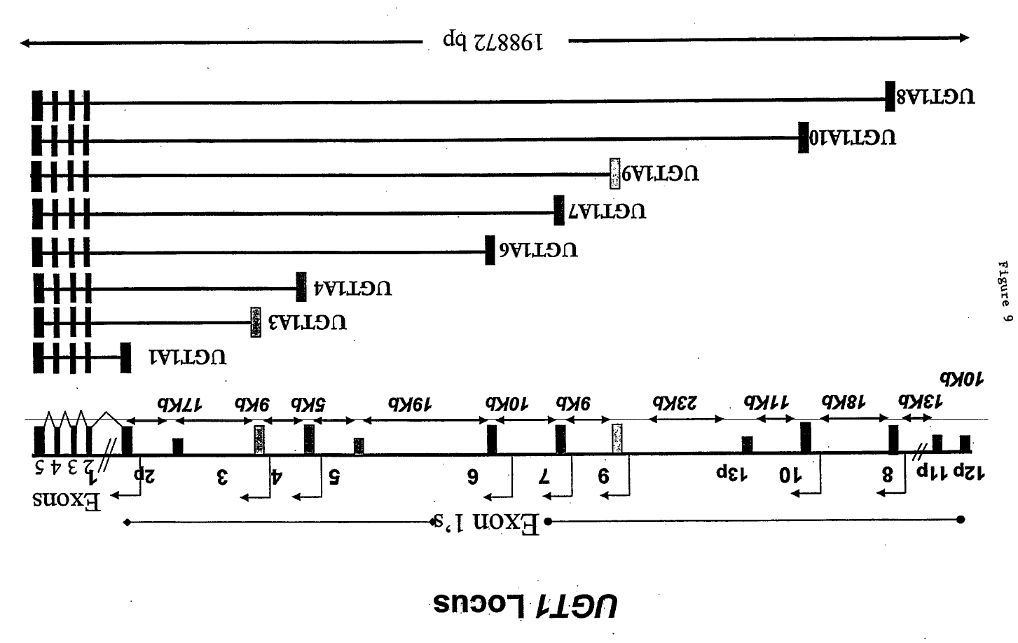

1: A non-human transgenic animal comprising
(a) at least one gene from a UDP-glucuronosyltransferase 1A (UGT1A) gene locust;
(b) the non-human transgenic animal of (a), comprising at least one UGT1A gene locus gene selected from the group consisting of UGT1A1, UGT1A3, UGT1A4, UGT1A6, UGT1A7, UGT1A8, UGT1A9 and UGT1A10l;
(c) the non-human transgenic animal of (a), wherein the non-human transgenic animal comprises at least one UGT1A gene locus exon as illustrated in FIG. 9;
(d) the non-human transgenic animal of (a) or (b), wherein the non-human transgenic animal comprises a complete UGT1A gene locus;
(e) the non-human transgenic animal of (a), wherein the non-human transgenic animal comprises at least one human gene from a human UDP-glucuronosyltransferase 1A (UGT1A) gene locus:
(f) the non-human transgenic animal of (a), wherein the UGT1A gene locus comprises a human UGT1A gene locus;
(g) the non-human transgenic animal of (a), wherein the UGT1A gene locus comprises a complete human UGT1A gene locus;
(h) the non-human transgenic animal of any one of (a) to (g), wherein the animal is a mouse;
(i) the non-human transgenic animal of any one of (a) to (g), wherein the animal is a goat, a rabbit, a sheep, a pig, a dog, a cow, a cat, a rat or a mouse; or
(j) the non-human transgenic animal of any one of (a) to (i), wherein the endogenous UDP-glucuronosyltransferase 1A (UGT1A) gene locus of the non-human transgenic animal is completely or partially disabled (“knocked out”).

2-10. (canceled)

11. A cell derived from the non-human transgenic animal of claim 1.

12. A cell line derived from the non-human transgenic animal of claim 1.

13. A tissue derived from the non-human transgenic animal of claim 1.

14. An isolated organ derived from the non-human transgenic animal of claim 1.

15. An inbred mouse line derived from the non-human transgenic animal of claim 1.

16. The inbred mouse line of claim 15, wherein the mouse line comprises a human UDP-glucuronosyltransferase 1A (UGT1A) gene locus.

17: A method of determining the pharmacokinetics, metabolism or toxicity of a compound comprising:
(i) (a) providing the non-human transgenic animal of claim 1;
(b) providing a test compound;
(c) administering the test compound to the transgenic animal; and
(d) determining the pharmacokinetics, metabolism or toxicity of the test compound in the non-human transgenic animal;
(ii) the method of (i), wherein the test compound comprises a drug, a small molecule, a polymer, a toxin, a steroid, a heme metabolite, a cosmetic, a lotion, a food, a food or dietary supplement, an herbicide, a pesticide, a pollutant or a natural product;
(iii) the method of (ii), wherein the toxin comprises an environmental toxin, a toxin derived from a natural product, a biological warfare agent or a toxin derived from a microorganism;
(iv) the method of (iii), wherein the environmental toxin is airborne, waterborne or a soil toxin;
(v) the method of any of (i) to (iv), wherein the test compound comprises a protein, a peptide, a nucleic acid, a carbohydrate, a polysaccharide, a fat, a steroid or a small molecule;
(vi) the method of any of (i) to (v), wherein the non-human transgenic animal is pregnant or pseudopregnant; or
(vii) the method of any of (i) to (vi), wherein the endogenous UGT1A gene locus of the non-human transgenic animal is partially or completed disabled (knocked out).

18-23. (canceled)

24: A method of determining if a compound induces or upregulates activity in a human UDP-glucuronosyltransferase 1A (UGT1A) gene locus comprising:
(i) (a) providing the non-human transgenic animal of claim 1;
(b) providing a test compound;
(c) administering the test compound to the transgenic animal; and
(d) measuring activity of the human UDP-glucuronosyltransferase 1A (UGT1A) gene locus in the non-human transgenic animal, cell, cell line, tissue or isolated organ, thereby determining if the test compound induced or upregulated activity in the non-human transgenic animal;
(ii) the method of (i), wherein the test compound comprises a drug, a small molecule, a polymer, a toxin, a steroid, a heme metabolite, a cosmetic, a lotion, a food, a food or dietary supplement, an herbicide, a pesticide, a pollutant or a natural product;
(iii) the method of (ii), wherein the toxin comprises an environmental toxin, a toxin derived from a natural product, a biological warfare agent or a toxin derived from a microorganism;
(iv) the method of (i), wherein the test compound comprises a protein, a peptide, a nucleic acid, a carbohydrate, a polysaccharide, a fat, a steroid or a small molecule;
(v) the method of any of (i) to (iv), wherein measuring activity of the human UDP-glucuronosyltransferase 1A (UGT1A) gene locus comprises measuring the chemical modification of the test compound;
(vi) the method of any of (v), wherein the chemical modification of the test compound to a hydrophilic glucuronide is determined; or
(vii) the method of any of (i) to (vi), wherein the non-human transgenic animal is pregnant or pseudopregnant.

25-30. (canceled)

31: A method of whether a compound is modified by the human UDP-glucuronosyltransferase 1A (UGT1A) gene locus pathway comprising:
(i) (a) providing the non-human transgenic animal of claim 1;
(b) providing a test compound;
(c) administering the test compound to the transgenic animal; and
(d) measuring the chemical modification of the test compound in the non-human transgenic animal;
(ii) the method of (i), wherein the test compound comprises a drug, a small molecule, a polymer, a toxin, a steroid, a heme metabolite, a cosmetic, a lotion, a food, a food or dietary supplement, an herbicide, a pesticide, a pollutant or a natural product;
(iii) the method of (ii), wherein the toxin comprises an environmental toxin, a toxin derived from a natural product, a biological warfare agent or a toxin, derived from a microorganism;
(iv) the method of (i), wherein the test compound comprises a protein, a peptide, a nucleic acid, a carbohydrate, a polysaccharide, a fat, a steroid or a small molecule;
(v) the method of any of (i) to (iv), wherein measuring activity of the human UDP-glucuronosyltransferase 1A (UGT1A) gene locus comprises measuring the chemical modification of the test compound;
(vi) the method of any of (v), wherein the chemical modification of the test compound to a hydrophilic glucuronide is determined; or
(vii) the method of any of (i) to (vi), wherein the non-human transgenic animal is pregnant or pseudopregnant.

32-36. (canceled)

37: A method of determining the pharmacokinetics, metabolism or toxicity of a compound comprising:
(i) (a) providing the cell of claim 11; (b) providing a test compound; (c) administering the test compound to the cell; and (d) determining the pharmacokinetics, metabolism or toxicity of the test compound in the cell;
(ii) the method of (i), wherein the test compound comprises a drug, a small molecule, a polymer, a toxin, a steroid, a heme metabolite, a cosmetic, a lotion, a food, a food or dietary supplement, an herbicide, a pesticide, a pollutant or a natural product;
(iii) the method of (ii), wherein the toxin comprises an environmental toxin, a toxin derived from a natural product, a biological warfare agent or a toxin derived from a microorganism;
(iv) the method of (iii), wherein the environmental toxin is airborne, waterborne or a soil toxin;
(v) the method of any of (i) to (iv), wherein the test compound comprises a protein, a peptide, a nucleic acid, a carbohydrate, a polysaccharide, a fat, a steroid or a small molecule;
(vi) the method of any of (i) to (v), wherein the endogenous UGT1A gene locus of the cell is partially or completed disabled (knocked out).

38: A method of determining the pharmacokinetics, metabolism or toxicity of a compound comprising:
(i) (a) providing the tissue of claim 13; (b) providing a test compound; (c) administering the test compound to the tissue; and (d) determining the pharmacokinetics, metabolism or toxicity of the test compound in the tissue;
(ii) the method of (i), wherein the test compound comprises a drug, a small molecule, a polymer, a toxin, a steroid, a heme metabolite, a cosmetic, a lotion, a food, a food or dietary supplement, an herbicide, a pesticide, a pollutant or a natural product;
(iii) the method of (ii), wherein the toxin comprises an environmental toxin, a toxin derived from a natural product, a biological warfare agent or a toxin derived from a microorganism;
(iv) the method of (iii), wherein the environmental toxin is airborne, waterborne or a soil toxin;
(v) the method of any of (i) to (iv), wherein the test compound comprises a protein, a peptide, a nucleic acid, a carbohydrate, a polysaccharide, a fat, a steroid or a small molecule;
(vi) the method of any of (i) to (v), wherein endogenous UGT1A gene locus of the tissue is partially or completed disabled (knocked out).

39: A method of determining the pharmacokinetics, metabolism or toxicity of a compound comprising:
(i) (a) providing the isolated organ of claim 14; (b) providing a test compound; (c) administering the test compound to the isolated organ; and (d) determining the pharmacokinetics, metabolism or toxicity of the test compound in the isolated organ;
(ii) the method of (i), wherein the test compound comprises a drug, a small molecule, a polymer, a toxin, a steroid, a heme metabolite, a cosmetic, a lotion, a food, a food or dietary supplement, an herbicide, a pesticide, a pollutant or a natural product;
(iii) the method of (ii), wherein the toxin comprises an environmental toxin, a toxin derived from a natural product, a biological warfare agent or a toxin derived from a microorganism;
(iv) the method of (iii), wherein the environmental toxin is airborne, waterborne or a soil toxin;
(v) the method of any of (i) to (iv), wherein the test compound comprises a protein, a peptide, a nucleic acid, a carbohydrate, a polysaccharide, a fat, a steroid or a small molecule;
(vi) the method of any of (i) to (v), wherein endogenous UGT1A gene locus of the isolated organ is partially or completed disabled (knocked out).

40: A method of determining if a compound induces or upregulates activity in a human UDP-glucuronosyltransferase 1A (UGT1A) gene locus comprising:
(i) (a) providing the cell of claim 11; (b) providing a test compound; (c) administering the test compound to the cell; and (d) measuring activity of the human UDP-glucuronosyltransferase 1A (UGT1A) gene locus in the cell, thereby determining if the test compound induced or upregulated activity in the cell;
(ii) the method of (i), wherein the test compound comprises a drug, a small molecule, a polymer, a toxin, a steroid, a heme metabolite, a cosmetic, a lotion, a food, a food or dietary supplement, an herbicide, a pesticide, a pollutant or a natural product;
(iii) the method of (ii), wherein the toxin comprises an environmental toxin, a toxin derived from a natural product, a biological warfare agent or a toxin derived from a microorganism;
(iv) the method of (i), wherein the test compound comprises a protein, a peptide, a nucleic acid, a carbohydrate, a polysaccharide, a fat, a steroid or a small molecule;
(v) the method of any of (i) to (iv), wherein measuring activity of the human UDP-glucuronosyltransferase 1A (UGT1A) gene locus comprises measuring the chemical modification of the test compound; or
(vi) the method of any of (v), wherein the chemical modification of the test compound to a hydrophilic glucuronide is determined.
